(19) United States
(12) Patent Application Publication
(10) Pub. No.: US 2010/0159458 A1
(43) Pub. Date: Jun. 24, 2010

(54) METHOD FOR PREDICTING THE RESPONSE TO A THERAPY

(75) Inventors: Heli Nevanlinna, Veikkola (FI); Rainer Fagerholm, Helsinki (FI); Johanna Tommiska, Espoo (FI)

Correspondence Address: MINTZ, LEVIN, COHN, FERRIS, GLOVSKY AND POPEO, P.C, ONE FINANCIAL CENTER, BOSTON, MA, 02111, US

(73) Assignee: Licentia Ltd., Helsinki (FI)

(21) Appl. No.: 12/516,052

(22) PCT Filed: Nov. 23, 2007
(86) PCT No.: PCT/FI07/50637

§ 371 Date: Jan. 6, 2010

(30) Foreign Application Priority Data
Nov. 24, 2006 (FI) 20061036
Apr. 27, 2007 (FI) 20075298

(51) Int. Cl. C12Q 1/68; C12Q 1/26

(52) U.S. Cl. 435/6; 435/25

(57) ABSTRACT

The present invention relates to cancer treatment and particularly to a method for predicting the response of a cancer subject to a given therapy. The invention provides a gene or gene product useful as a predictive marker for classifying the subjects. Also disclosed are diagnostic tools, test kits and compositions and their use in the method. The invention is based on the use of NAD(P)H:Quinone oxidoreductase 1, NQO1, which enables the identification and classification of subjects who would benefit from being excluded from a treatment, particularly from anthracycline-based adjuvant chemotherapy with epirubicin.

### FIELD OF THE INVENTION

The present invention relates to cancer treatment and particularly to a method for selecting a cancer therapy and predicting the response of a subject to a given therapy. The invention provides a gene or gene product useful as a predictive marker for classifying the subjects. The invention is based on the detection of NAD(P)H:Quinone oxidoreductase, NQO1, polymorphism, which enables the identification and classification of subjects who would benefit from being excluded from a treatment, particularly from anthracycline-based adjuvant chemotherapy with epirubicin.

### BACKGROUND OF THE INVENTION

Cancer is a class of diseases or disorders where division of cells is uncontrolled and cells are able to spread, either by direct growth into adjacent tissue through invasion, or by implantation into distant sites by metastasis. Cancer can be treated by surgery, chemotherapy, radiation therapy, immunotherapy, monoclonal antibody therapy or combination thereof or other methods. The choice of therapy depends upon the location and grade of the tumor and the stage of the disease, as well as the general state of the patient. Generally, cancer patients can be effectively treated using these conventional methods, but exceptions exist and some of the current therapies are known to be ineffective or may even induce serious side effects which diminish the quality of life of the patients.

No tumor factors are presently available in clinical use which would predict response to chemotherapy. For example markers for breast cancer do not specifically give information whether a certain treatment is suitable for a patient. Presently, the treatment is aimed to be applied as early as possible and not only curatively. To improve the outcome of individual cancer therapies, there is a great demand for new biomarkers, which would enable identification of subsets of patients who benefit from a given treatment regimen and those who do not.

Breast cancer is the most common cancer type among women worldwide, and the second leading cause of death. The prognosis of patients is influenced by the tumor stage, grade, HER2 (ERBB2) and hormonal receptor status, which are used to classify the tumor and to choose the individual treatment regimen for each patient (Goldhirsch et al., 2001). Of these factors only hormone receptor status and HER2-expression predict an improved response to treatment with endocrine therapy and monoclonal antibody immunotherapy with Trastuzumab, respectively. There is a great demand for tumor factors, which would predict response to chemotherapy. Very recently, HER2 amplification was suggested to associate with clinical responsiveness to anthracycline-containing chemotherapy (Pritchard et al., 2006).

NAD(P)H:quinone oxidoreductase (NQO1, NAD(P)H:menadione oxidoreductase, DT-diaphorase) is a phase II detoxification enzyme implicated in cellular protection against oxidative stress and carcinogenesis, including scavenging of superoxides (Siegel et al., 2004), maintenance of lipid-soluble antioxidants and reduction of toxic quinones to less toxic excretable hydroquinones (Beyer et al., 1996; Siegel et al., 1997; Winski et al., 2001), as well as stabilization of the key tumor suppressor protein p53 (Anwar et al., 2003; Asher et al., 2001; Asher et al., 2002a; Asher et al., 2002b). NQO1 deficient mice show reduced p53 induction and apoptosis and increased susceptibility to chemically induced tumors (Iskander et al., 2005; Long et al., 2000). Furthermore, such mice have impaired immune response (Iskander et al., 2006) and NF-κB function (Ahn et al., 2006). The p53 pathway is the most important known mechanism of cellular defense against carcinogenesis, and a major fraction of human cancers contain mutations in the p53 gene that generate a dysfunctional or absent protein (Kastan 2007).

The normal form of the NQO1 gene is designated as polymorphic form NQO1*1. NQO1*2 polymorphism differs from NQO1*1 as follows. NQO1*2 allele represents a cytosine to thymine substitution at position 609 (C609T) in the cDNA (NCBI sequence ID:J03934.1, refSNP ID:rs1800566) coding for a proline to serine change at position 187 (Pro187Ser) of the protein. The polymorphism is homozygous in 4-20% of human population, depending on ethnicity (Kelsey et al., 1997; Nioi et al., 2004). Homozygous carriers of c.609C>T allele have no measurable NQO1 activity. Correlation between susceptibility to tumors and the polymorphism in NQO1 gene or its gene products has been described, but no methods for predicting the response to specific cancer or tumor therapies have so far been demonstrated. The NQO1*3 polymorphism differs from normal NQO1 gene in that nucleotide residue 465 is changed from cytosine to thymine (c.465C>T), resulting in a change at amino acid residue 139 from arginine to tryptophan (R139W). The NQO1*3 polymorphism is very rare.

NQO1*2 homozygous individuals are sensitive to benzene hematotoxicity and susceptible to subsequent acute nonlymphocytic leukemia (Garte et al., 2005; Rothman et al., 1997), and they show increased risk of cancer, particularly leukemias (Krajinovic et al., 2002a; Larson et al., 1999; Naoe et al., 2000; Smith et al., 2001; Wiemels et al., 1999). The NQO1*2 variant also associates with an increased risk of relapse or death among children undergoing treatment for childhood acute lymphocytic leukemia (Krajinovic et al., 2002b). It is suggested that the NQO1*2 polymorphism is relevant to response to induction therapy in patients with acute myeloid leukemia (Barragan et al. 2007). Moreover, recent meta-analysis data suggest that NQO1 genotype affects susceptibility to lung, bladder and colorectal cancer, depending on ethnicity and smoking status (Chao et al., 2006). Several studies have also addressed the association between NQO1 status and breast cancer risk (Fowke et al., 2004; Menzel et al., 2004; Sarmanova et al., 2004), but on a scale insufficient to reach definite conclusions. No significant effect on overall survival in breast cancer has been previously detected (Goode et al., 2002). Goldberg et al. 1998 and Fleming et al. 2002 have studied the role of NQO1 gene to mitomycin C (MMC) response. Ross et al. 2000 review the enzymatic role of NQO1 and define the regulation and function of NQO1 gene. Shi et al. 1999 describe methods for analysis of NQO1*2 polymorphism.

WO 2005/119260 discloses a method for monitoring a response to chemotherapy in breast cancer patients by measuring expression levels of specific gene products e.g. NQO1 before and after the onset of chemotherapy. A change in the expression level is used to estimate the effect of chemotherapy. The measurement of an expression level of a gene from a tumor sample indicates the progress of the cancer treatment at a certain state in a certain tissue. The method is quantitative and several samples are required in order to determine the change in the expression level. US 20010034023 discloses a method utilizing variance in genes relating to drug processing e.g. in NQO1 for selecting a drug treatment for patients suffering from a disease. WO 2005/098037, WO 2004058153, WO 2006035273 and US 2003158251 describe the use of NQO1 gene as a marker. WO 02052044 discloses methods for identifying gene variations related to drug metabolism. WO 2005/024067 discloses a genetic analysis for stratification of breast cancer risk.

It is presently acknowledged that a significant number of treated patients do not benefit from the therapies generally applied as a first choice. The delay in applying an effective, curative treatment causes unnecessary pain and discomfort to patients and may even be fatal, and it is not cost-effective for the society. Methods for early identification and classification of the subjects who will probably not benefit from a costly, but ineffective treatment and for whom an alternative treatment regimen is needed, are urgently required in order to provide more cost-effective and curative therapies.

### SUMMARY OF THE INVENTION

The present invention aims at an improved, individualized therapy, by using biomarkers, which enable the identification of subjects who profit most from a given treatment and those who would benefit from being excluded from a given treatment. These predictive markers would be highly beneficial and would significantly reduce the side-effects and costs caused by ineffective treatment and allow a faster presentation to alternative, more effective therapies.

The present invention is based on the surprising finding that it is possible based on the presence of a mutant or non-functional NQO1 gene or gene product, or absence of a normal or functional NQO1 gene or gene product to determine whether a subject would benefit from being excluded from a given treatment regimen. Especially it has been shown that homozygous cytosine to thymine substitution at position 609 in the polynucleotide sequence NCBI sequence ID:J03934.1, ref SNP IDS:rs1800566, named also c.609C>T allele or NQO1*2 polymorphism, resulting in the change of proline to serine (P187S) in an encoded gene product, is associated with poor survival among breast cancer patients, especially after anthracycline-based adjuvant chemotherapy with epirubicin (FEC). Also other variations, such as alterations, deletions, insertions or replacements of one or more nucleotides, or also epigenetic changes, causing that the subject or the tumor is not capable of producing a normal or functional gene product, can be used for identifying subjects that would benefit from being excluded from cancer therapy. The polymorphism of NQO1 and its association to cancers was previously known, but the results of the present inventors demonstrated for the first time the prognostic and predictive value of NQO1 polymorphism for screening the group of subjects that would benefit from being excluded from a given treatment regimen. The method of the invention enables the determination by genotyping before the onset of the chemotherapy, especially anthracyclin based chemotherapy, whether the patient would benefit from said therapy. The patients with the NQO1 gene variation do not benefit from the said treatment and their condition may even be impaired.

The present invention is related to a method for selecting a cancer therapy based on subject's genetic background, wherein the detection of presence of a mutant or non-functional NQO1 gene or gene product, or absence of a normal or functional NQO1 gene or gene product in a sample of said subject, allows a classification of the subjects in at least two subsets, one which may be treated with cancer therapy and another who would benefit from being excluded from said cancer therapy. An alternative therapy could be considered to the subjects of the second subset.

The present invention is related to a method for selecting a cancer therapy based on subject's genetic background, wherein the method comprises the steps of determining the presence of a mutant or non-functional NAD(P)H:Quinone oxidoreductase 1, NQO1, gene or gene product, or absence of a normal or functional NQO1 gene or gene product from a sample of the subject comprising healthy or tumor cells before the onset of a chemotherapy, wherein said NQO1 gene carries a change in a nucleotide sequence; and classifying subjects in at least two subsets wherein one subset having a normal or functional NQO1 gene may be treated with cancer therapy and another subset having a mutant or non-functional NQO1 gene would benefit from being excluded from said cancer therapy.

The present invention is related to a method, wherein the absence of a normal or functional NQO1 gene or gene product from the sample of the subject due to homozygous, hemizygous or other genetic or genomic alterations indicates that the subject would benefit from being excluded from said cancer therapy. An alternative therapy could be considered.

The present invention is related to a method, wherein the NQO1 gene carries a change of one or more nucleotides, which results in a non-functional NQO1 gene or gene product.

The present invention is related to a method, wherein the NQO1 gene carries a change in the nucleotide sequence corresponding to the cytosine to thymine substitution at position 609 of the polynucleotide sequence in NCBI sequence ID:J03934.1 or refSNP ID:rs1800566 set forth in SEQ ID NO:4 comprising a c.609C>T allele or NQO1*2 polymorphism, thereby resulting in the amino acid change of proline to serine at position 187, P187S, of the encoded gene product.

The present invention is related to a method, wherein the NQO1 gene in the tumor cells is non-functional or the normal gene or gene product is absent due to homozygous, hemizygous or other genetic or genomic alterations.

The present invention is also related to a method, wherein a change in the nucleotide sequence is in linkage disequilibrium to position 609 of the polynucleotide sequence in NCBI sequence ID:J03934.1 or refSNP ID:rs1800566 set forth in SEQ ID NO:4 or to any other change of one or more nucleotides in said polynucleotide sequence resulting in a similar functional effect.

The present invention is also related to a method, wherein two copies of the c.609C>T allele are present in the subject indicating that the subject is a homozygous carrier of the c.609C>T allele and benefits from being excluded from cancer therapy.

The present invention is also related to a method, wherein one copy of the c.609C>T allele is present in the tumor with loss or inactivation of the other allele indicating that the tumor cells are hemizygous for the c.609C>T allele and the subject benefits from being excluded from the cancer therapy.

The present invention is also related to a method, wherein the method comprises determining the identity of nucleotides in the nucleotide position c.609; and classifying the subject to a subset having a mutant or non-functional NQO1 gene if the T allele is present in both copies in the c.609 position, and to a subset having a normal or functional NQO1 gene if one of the alleles present in the c.609 position is C.

The presence or absence of said normal or functional gene and its gene products can be determined by using a multitude of detection methods based on the detection of polynucleotides including DNA or RNA, or proteins or polypeptides in question as demonstrated by in vitro detection of a c.609C>T allele or NQO1*2 polymorphism in the NQO1 gene resulting in the P187S change in a gene product. As more information about the human genome is accumulating and it can be expected that the genome of a subject has been previously determined and available, the therapy can be determined based on the known genotype of the subject presenting with a certain type of cancer.

The presence of a normal or functional NQO1 gene or gene product indicates that the subject most probably profits from anthracycline-based adjuvant chemotherapy. Presence of two copies of the c.609C>T allele (homozygosity) indicates no response to the therapy or even a detrimental effect of the therapy. This applies also to tumor hemizygosity, wherein one copy of an allele can be lost in tumors because of the loss of heterozygosity, because of inactivation due to epigenetic mechanisms or because of somatic mutations. Presence of one copy of the c.609C>T allele in the tumor with loss or inactivation of the other allele indicates that the tumor cells are hemizygous for the c.609C>T allele and the subject benefits from being excluded from the treatment. Heterozygosity may cause decreased functionality.

A subset of subjects carrying a single nucleotide substitution in the NQO1 gene, resulting in a change of one amino acid in the amino acid sequence of the encoded gene product, said change having an effect on the NQO1 function, would benefit from being excluded from said cancer therapy, wherein said cancer therapy comprises chemotherapy.

The present invention is related to a method wherein, the chemotherapy is carried out with a chemotherapy agent, which comprises a topoisomerase II inhibitor. The topoisomerase II inhibitor comprises amsacrine, mitoxantrone, piroxantrone, dactinomycin, anthracyclins, or epipodofyllotoxin-derivative or derivatives thereof. The anthracyclins comprise doxorubicin, daunorubicin, idarubicin, aclarubicin or epirubicin or derivatives thereof. The present method is particularly useful when the treatment or cancer therapy comprises anthracycline-based adjuvant chemotherapy and more particularly with epirubicin or derivatives thereof.

The present invention relates to a method, wherein the cancer therapy may comprise early curative therapy. The early curative therapy means the treatment, which is the first therapy given to a subject in need. The present invention relates to a method, wherein the cancer therapy comprises treatment of metastatic cancer.

The method may be used for predicting the response of subjects suffering from a cancer or a malignancy, comprising either primary or metastatic tumor, wherein said cancer or malignancy is breast cancer, lung, bladder, prostatic, ovarian, pancreatic, gastric or colorectal cancer, cancer of the large intestine, non-Hodgkin's lymphoma, head neck cancer, large cell lung carcinoma, small cell lung carcinoma or soft tissue sarcoma or children's tumor. Said cancers of malignancies can be treated with anthracyclin-based adjuvant chemotherapy. The method is particularly useful for predicting responses from subjects suffering from breast cancer.

The present method is particularly useful for breast cancer patient homozygous for the c.609C>T allele or NQO1*2 polymorphism of NQO1 gene, or any other change of one or more nucleotides in said polynucleotide sequence resulting in a similar functional effect, or a patient having tumor cells hemizygous for the c.609C>T allele or NQO1*2 polymorphism, or any other change of one or more nucleotides in said polynucleotide sequence resulting in a similar functional effect. In these cases the subject would benefit from being excluded from a planned treatment using anthracycline-based adjuvant chemotherapy with epirubicin.

One subgroup of subjects for whom the method is advantageous is a breast cancer patient heterozygous for the c.609C>T allele or NQO1*2 polymorphism or any other change of one or more nucleotides resulting in a similar functional effect of NQO1 gene and wherein the cancer comprises a p53 immunopositive tumor and said cancer therapy is an anthracyclin-based adjuvant chemotherapy.

The method of the present invention relates to an in vitro method, wherein isolated and purified polynucleotide sequences or fragments thereof from a cell or tissue sample of a subject or an in vitro sample lysate from a subject comprising said polynucleotide sequences or fragments thereof, including DNA or RNA, or isolated and purified proteins or fragments thereof from a cell or tissue sample of a subject or an in vitro sample lysate from a subject comprising said proteins or fragments thereof, are determined by per se known techniques. The sample comprises a DNA, or RNA, or a protein or a fragment thereof, originating from the subject and representing an inherited genotype or phenotype of the subject, or a genotype of a tumor.

The method of the present invention comprises any conventional genotyping method or phenotyping method or any method based on DNA, RNA or amino acid. A useful genotyping method based on DNA or RNA comprises a technique for single nucleotide polymorphism (SNP) detection and genotyping, such as restriction fragment length polymorphism PCR (RFLP-PCR), single strand conformation polymorphism (SSCP), allele specific hybridization, primer extension, allele specific oligonucleotide ligation or sequencing. The method of the present invention applies the genotyping method based on DNA or RNA sequence specificity comprising identification of the c.609C>T allele or NQO1*2 polymorphism in the NQO1 gene.

The method of the present invention applies the phenotyping method comprising detection of lack of the NQO1 gene product due to the polymorphism or any other genetic or genomic alteration in NQO1 gene. The method of the present invention applies the phenotyping method based on identification of the P187S mutation in the NQO1 gene product. The present invention is related to a method for providing a more effective treatment for a subject suffering from cancer, wherein the absence of a normal or functional NQO1 gene or gene product indicates that the subject is excluded from a cancer treatment.

The present invention is related to a method for treating a subject suffering from cancer or malignancy, comprising determining the presence of a mutant or non-functional NQO1 gene or gene product, or absence of a normal or functional NQO1 gene or gene product from a sample of the subject; and determining the proper therapy for said subject based on results of the genotype determination, wherein in the absence of a normal or functional NQO1 gene the subject is excluded from a cancer therapy.

The present invention is related to a method for optimizing clinical trial design for selecting a cancer therapy based on subject's genetic background, wherein the method comprises determining the presence of a mutant or non-functional NQO1 gene or gene product, or absence of a normal or functional NQO1 gene or gene product from a sample of the subject; and allowing classification of the subjects in at least two subsets, wherein one subset having a normal or functional NQO1 gene may be treated with cancer therapy and another subset having a mutant or non-functional NQO1 gene would benefit from being excluded from said cancer therapy.

The present invention is related to a method for selecting a cancer therapy for treatment of metastatic cancer based on subject's genetic background, wherein the method comprises the steps of determining the presence of a mutant or non-functional NQO1 gene or gene product or absence of a normal or functional NQO1 gene or gene product from a sample of the subject comprising healthy or tumor cells wherein said NQO1 gene carries a change in a nucleotide sequence; and classifying subjects in at least two subsets wherein one subset having a normal or functional NQO1 gene may be treated with cancer therapy and another subset having a mutant or non-functional NQO1 gene would benefit from being excluded from said cancer therapy.

The subject may have been treated with any cancer therapy to cure a primary tumor. The genotyping of determining the presence of a mutant or non-functional NQO1 gene, or absence of a normal or functional NQO1 gene from a sample of the subject comprising healthy or tumor cells is carried out. may have been done before the detection of metastasis. The determination is done before the onset of chemotherapy to determine whether the subject would benefit from the intended therapy such as anthracyclin based chemotherapy. The time frame between the treatments may vary up to several years.

### BRIEF DESCRIPTION OF THE DRAWINGS

In the following the invention will be described in greater detail by means of embodiments with references to the attached figures.

FIG. 1 demonstrates that NQO1*2 genotype associates with reduced cumulative survival in breast cancer, particularly among subgroups stratified by p53 immunohistochemistry and adjuvant FEC treatment status. Comparisons of Kaplan-Meier survival curves between NQO1*2 (P187S) genotypes among selected groups of patients are presented: n=number of cases; p=p-value of log-rank test; CS_{5y}=cumulative survival after five years of follow-up (confidence intervals given in parentheses). The labels beside the curves denote NQO1 (P187S) genotype. PP=lines homozygous for normal NQO1: NQO1 001 (NQO1*1), PS=heterozygous variant NQO1 003 and SS=LBL51 (NQO1*2) lacking functional NQO1.

FIG. 1a depicts overall survival after first breast cancer diagnosis among all valid cases, including both familial and unselected patients. Consistent with the level of detectable NQO1 protein seen in cell lines (FIG. 5a), the survival-curve of NQO1 heterozygotes closely resembled that of wild-type homozygotes. To maximize statistical power, the wild-type homozygotes (PP) and heterozygous (PS) patients were grouped together in subsequent analyses.

FIG. 1b depicts overall survival among patients who received endocrine therapy; FEC-treated patients have been excluded from this group.

FIG. 1c depicts overall survival among patients with p53 immunopositive tumors.

FIG. 1d depicts overall survival among patients with p53 immunonegative tumors.

FIG. 1e depicts overall survival among patients who received adjuvant FEC treatment.

FIG. 1f depicts overall survival among patients who received non-anthracycline based treatment or no treatment.

FIG. 2 demonstrates NQO1 genotype and p53 status impact on sensitivity to epirubicin in cultured human cells.

FIG. 2a depicts proliferative activity of MCF7DT9 overexpressing NQO1 and the vector control MCF7neo6 cell lines, determined by MTT-like AlamarBlue assay. Cells were treated with increasing concentrations of epirubicin for 72 h. MCF7DT9 are significantly more sensitive to epirubicin than MCF7neo6 cells (p<0.001).

FIG. 2b depicts Sytox green/Hoechst viability assay of MCF7DT9 and MCF7neo6 cells. Viability was assessed at 72 h of epirubicin treatment by fluorescent microscopy. Higher amounts of dead cells (significantly higher after treatment with 100 and 200 ng/ml epirubicin (p=0.05 and p=0.015, respectively)) are observed in the MCF7DT9 cell line.

FIG. 2c depicts proliferative activity of B-cell lymphoblast cell lines homozygous for normal NQO1: NQO1 001 (NQO1*1, PP), heterozygous variant NQO1 003 (PS) and LBL51 (NQO1*2, SS) lacking functional NQO1, at 48 h of treatment with increasing concentrations of epirubicin. NQO1*1 cells are more sensitive to epirubicin than NQO1*2 (significantly more sensitive after treatment with 25 ng/ml of epirubicin and higher doses (25 ng/ml: p=0.003, 50 ng/ml: p=0.01, 250 ng/ml: p=0.005, 500 ng/ml: p=0.0001, respectively)).

FIG. 2d depicts Sytox green/Hoechst viability assay of B-cell lymphoblast cell lines at 48 h of epirubicin treatment. Significantly higher amount of dead cells in NQO1*1 cells after treatment with 25 ng/ml epirubicin (p=0.02).

FIG. 2e depicts Western blotting analysis of PARP cleavage in MCF7DT9 and neo6 cell lysates harvested at the indicated times of epirubicin treatment (100 ng/ml).

FIG. 2f shows that lack of functional NQO1 reduces epirubicin-induced PARP-cleavage, and NQO1*1 (P/P) normal cells have higher initial levels of p53 and p21 than cells lacking NQO1. Western blotting analysis of B-cell lymphoblast cell lysates harvested at the indicated times of epirubicin treatment (100 ng/ml).

FIG. 3 demonstrates that p53 affects NQO1-mediated cell death induced by epirubicin but not by tumor necrosis factor α (TNF).

FIG. 3a depicts that proliferative activity of MCF7 cells was measured 72 h of treatment with increasing doses of TNF. MCF7DT9 are significantly more sensitive to TNF (20 ng/ml) than neo6 cells (p=0.008).

FIG. 3b is an immunoblotting analysis of NQO1 expression levels in U2OS-p53DD cells transfected with pEFIRES-NQO1 (EFNQ13) or pSUPER-NQO1 (NQ12).

FIG. 3c depicts proliferative activity of U2OS-p53DD cells overexpressing NQO1 (stably transfected with pEFIRES-NQO1) with (p53DD silenced) or without tetracycline (p53DD expressed) in response to increasing concentrations of epirubicin for 48 h.

FIG. 3d depicts proliferative activity of U2OS-p53DD cells transfected with pSUPER-NQO1 (shRNA plasmid) in response to epirubicin at 48 h of treatment.

FIG. 3e depicts proliferative activity of U2OS-p53DD cells overexpressing NQO1 (stably transfected with pEFIRES-NQO1) with (p53DD silenced) or without tetracycline (p53DD expressed) in response to TNF at 72 h of treatment.

FIG. 3f depicts proliferative activity of U2OS-p53DD cells transfected with pSUPER-NQO1 (shRNA plasmid) in response to TNF at 72 h of treatment.

FIG. 3g depicts proliferative activity of the p53-deficient breast cancer cell lines MDA MB157 (NQO1*1, PP) and MDA MB231 (NQO1*2, SS) in response to treatment with increasing concentrations of epirubicin.

FIG. 3h depicts proliferative activity of the p53-deficient breast cancer cell line MDA MB231-NQO1 in response to treatment with increasing concentrations of epirubicin.

FIGS. 3i and 3k depict proliferative activity of the p53-deficient breast cancer cell lines MDA MB157 (NQO1*1, PP) and MDA MB231 (NQO1*2, SS) and MDA MB231-NQO1 (i) in response to treatment with increasing concentrations of TNF at 72 h of treatment. NQO1 proficient cells are significantly more sensitive to TNF treatment (i: p<0.0001 after 10 and 20 ng/ml TNF; k: p=0.024 after 10 ng/ml TNF).

FIG. 4 demonstrates activity of the NF-κB pathway as well as responses of human breast cancer cell lines to diverse treatments and a schematic model of pathways involved in the tumor responses to epirubicin and TNF.

FIG. 4a shows that epirubicin but not methotrexate induces DNA damage response. MCF7 neo6 and DT9 cells were treated with methotrexate for different duration (or 24 h of epirubicin as a positive control) and harvested at the indicated times. Immunoblotting analysis was performed for proteins involved in the DNA damage response: γ-H2AX, p53 (and p53-Ser15-P) and p21.

FIG. 4b depicts that combined treatment with TNF and epirubicin activates proliferation in NQO1*2 p53mut breast cancer cells. MDA MB231 and MCF7 DT9 cells were treated with either TNF (10 ng/ml) or epirubicin (50 ng/ml) or with the combination. Proliferative activity was measured after 72 h of treatment.

FIG. 4c depicts schematic model of NQO1-associated induction of cell death by epirubicin and TNF, and the relative impact of NQO1 and/or p53 defects on breast cancer response to treatment. NQO1 stabilizes p53 and enhances epirubicin- and TNF-induced apoptosis in a NQO1*1 and p53 wt background. Loss of function of NQO1 or p53 (crossed symbols) lead to reduced treatment response to epirubicin and TNF in vitro, impaired NF-κB signaling and reduced p53-dependent and independent cell death after treatment. Full arrows represent functional pathways contributing to cell death, full lines with a blocking bar represent pathways that promote survival and proliferation, and dashed lines show inactive pathways. The narrowing and widening horizontal panels under the pathways indicate, respectively, the reduced cell death and likely increasing oxidative stress and genomic instability associated with the indicated combinations of p53 and NQO1 defects. There is also a functional cross-talk between the parallel p53- and NF-κB pathways (Janssens et al., 2006) (see Detailed description of the invention for further details).

FIG. 4d depicts that nuclear translocation of NF-kB/p65 is induced in response to epirubicin (100 ng/ml), TNF (10 ng/ml) or the combination in MCF7 neo6 and DT9 cells at the indicated time after treatment. Note the nuclear localization that is particularly enhanced after combined treatment in the NQO1 overexpressing MCF7DT9 cells.

FIG. 4e depicts that the NF-κB-pathway is activated in a subset of breast cancer patients even before initiation of adjuvant chemotherapy. Immunohistochemical staining for the p65 subunit of NκkB; From left to right: normal human breast tissue, invasive ductal carcinoma, comedo type carcinoma in situ, and invasive ductal carcinoma of the breast. Note the cytoplasmic localisation of p65 in normal breast and the first carcinoma, in contrast to preferentially nuclear staining pf p65 in the latter two tumors. Representative pictures of breast tissue are shown.

FIG. 5a demonstrates immunoperoxidase staining for NQO1 protein in human cell lines. Left from top to bottom are the breast cancer cell lines: MDA-MB157 (PP), MCF-7 (PS) and MDA-MB231 (SS); on the right the lymphoblastoid cell lines: NQO1 002 (PP), LBL47 (PS) and LBL51 (SS). No NQO1 expression is observed in either of the SS homozygous cell lines.

FIG. 5b demonstrates that NQO1 PS heterozygotes have reduced survival among patients with p53 immunopositive tumors. PP, PS and SS denote NQO1 P187S genotypes. n=number of valid cases; p(trend)=significance of the linear trend towards worse survival according to increasing number of NQO1*2 alleles (Kaplan-Meier trend test as implemented in SPSS 12.0).

FIG. 6 discloses that NQO1*2 homozygous patients have reduced survival after breast cancer metastasis.

### DETAILED DESCRIPTION OF THE INVENTION

### Abbreviations

NQO1 NAD(P)H:Quinone oxidoreductase 1
PP homozygous for normal NQO1: NQO1 (NQO1*1)
PS heterozygous variant NQO1:NQO1*2
SS homozygous for NQO1*2 (lacking functional NQO1)

### DEFINITIONS

Unless otherwise specified, the terms used in the present invention, have the meaning commonly used in the medical science and cancer research. Some terms, however, may be used in a somewhat different manner and some terms benefit from additional explanation to be correctly interpreted for patent purposes. Therefore some of the terms are explained in more detail below.

A term “based on subject's genetic background” means that the subject's genetic map is known or is determined from a sample. Especially the sequence of NQO1 gene is known or determined.

A “polymorphic site” or “polymorphism site” or “polymorphism” is the locus or position within a given sequence at which divergence occurs. A “polymorphism” refers to the occurrence of two or more forms of a gene or position within a gene (allele), in a population. A “polymorphic locus” is a marker or site at which divergence from a reference allele occurs. The phrase “polymorphic loci” is meant to refer to two or more markers or sites at which divergence from two or more reference alleles occurs. Preferred polymorphic sites have at least two alleles, each occurring at frequency of greater than 1%, and more preferably greater than 10% or 20% of a selected population. A polymorphic site may be at known positions within a nucleic acid sequence or may be determined to exist using the methods described below. Polymorphisms may occur in both the coding regions and the noncoding regions of genes. A polymorphic locus may be as small as one base pair. Polymorphic loci include single-nucleotide polymorphism sites (SNPs), restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the “reference form” or “reference allele” and other allelic forms are designated as alternative forms or “variant alleles”. The allelic form occurring most frequently in a selected population is sometimes referred to as the wild type form. Diploid organisms may be homozygous or heterozygous for allelic forms. A diallelic or biallelic polymorphism has two forms. A triallelic polymorphism has three forms.

For the purposes of the present invention the terms “polymorphic position”, “polymorphic site”, “polymorphic locus”, and “polymorphic allele” shall be construed to be equivalent and are defined as the location of a sequence identified as having more than one nucleotide represented at that location in a population comprising at least one or more individuals, and/or chromosomes. A polynucleotide sequence may or may not comprise one or more polymorphic loci.

As used herein, “linkage” describes the tendency of genes, alleles, loci or genetic markers to be inherited together as a result of their location on the same chromosome. It can be measured by percent recombination between the two genes, alleles, loci or genetic markers. In general “linkage” as used in population genetics, refers to the co-inheritance of two or more nonallelic genes or sequences due to the close proximity of the loci on the same chromosome, whereby after meiosis they remain associated more often than the 50% expected for unlinked genes.

As used herein, the term “genotype” is meant to encompass the particular allele present at a polymorphic locus of a DNA sample, a gene, and/or chromosome. A “genotype” is defined as the genetic constitution of an organism, usually in respect to one gene or few genes or a region of a gene relevant to a particular context i.e. the genetic loci responsible for a particular phenotype. A region of a gene can be as small as a single nucleotide in the case of a single nucleotide polymorphism.

“Genotyping” means the process of determining the genotype of an individual with a biological assay. Sequence specific genotyping method means any method based on DNA, RNA or amino acid sequence specificity. Examples of such sequence specific genotyping methods include but are not limited to a technique for single nucleotide polymorphism (SNP) detection and genotyping, such as restriction fragment length polymorphism PCR (RFLP-PCR), SSCP, allele specific hybridization, primer extension, allele specific oligonucleotide ligation or sequencing. Determining of genotype may also include one or more of the following techniques, restriction fragment length analysis, sequencing, micro-sequencing assay, hybridization, invader assay, gene chip hybridization assays, oligonucleotide ligation assay, ligation rolling circle amplification, 5′ nuclease assay, polymerase proofreading methods, allele specific PCR, matrix assisted laser desorption ionization time of flight (MALDI-TOF) mass spectroscopy, ligase chain reaction assay, enzyme-amplified electronic transduction, single base pair extension assay and reading sequence data. “Single nucleotide polymorphisms (SNPs)” are DNA sequence variations that occur when a single nucleotide (A, T, C, or G) in the genome sequence is changed, which occur approximately once every 100 to 300 bases. A single nucleotide polymorphism usually arises due to substitution of one nucleotide for another at the polymorphic site.

The existence of NQO1 polymorphism can be assessed by any known method for polymorphism detection. Such methods include sequencing based methods, hybridization based methods and primer extension methods as described above.

A “phenotype” refers to the observable characters of an organism.

As used herein, the term “haplotype” is meant to encompass the combination of genotypes across two or more polymorphic loci of a DNA sample, a gene, and/or chromosome, wherein the genotypes are closely linked. A “haplotype” is a set of alleles situated close together on the same chromosome that tend to be inherited together. A combination of genotypes may be inherited together as a unit, and may be in “linkage disequilibrium” relative to other haplotypes and/or genotypes of other DNA samples, genes, and/or chromosomes.

As used herein, the term “linkage disequilibrium” refers to a measure of the degree of association between two alleles in a population. For example, when alleles at two distinctive loci occur in a sample more frequently than expected given the known allele frequencies and recombination fraction between the two loci, the two alleles may be described as being in “linkage disequilibrium”.

As used herein, the terms “genotype assay” and “genotype determination”, and the phrase “to genotype” or the verb usage of the term “genotype” are intended to be equivalent and refer to assays designed to identify the allele or alleles at a particular polymorphic locus or loci in a DNA sample, a gene, and/or chromosome. Such assays may employ single base extension reactions, DNA amplification reactions that amplify across one or more polymorphic loci, or may be as simple as sequencing across one or more polymorphic loci. A number of methods are known in the art for genotyping, with many of these assays being described herein or referred to herein.

A “single nucleotide polymorphism” (SNP) occurs at a polymorphic locus occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic locus. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic locus is occupied by a base other than the reference base. For example, where the reference allele contains the base “T” at the polymorphic site, the altered allele can contain a “C”, “G” or “A” at the polymorphic locus. By altering amino acid sequence, “SNPs” may alter the function of the encoded proteins. The discovery of the SNP facilitates biochemical analysis of the variants and the development of assays to characterize the variants and to screen for pharmaceutical compounds that would interact directly with one or another form of the protein. SNPs (including silent SNPs) may also alter the regulation of the gene at the transcriptional or post-transcriptional level. SNPs (including silent SNPs) also enable the development of specific DNA, RNA, or protein-based diagnostics that detect the presence or absence of the polymorphism in particular conditions.

An “allele” is defined as any one or more alternative forms of given gene at a particular locus on a chromosome. Different alleles produce variation in inherited characteristics. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if they are genetically different the cell or organism is said to be “heterozygous” with respect to the particular gene. When “genes” are considered simply as segments of a nucleotide sequence, allele refers to each of the possible alternative nucleotides at a specific position in the sequence.

A “polynucleotide sequence” can be DNA or RNA in either single- or double-stranded form. A polynucleotide sequence can be naturally occurring or synthetic or semisynthetic, but is typically prepared by synthetic or semisynthetic means, including PCR. As used herein, a “polynucleotide” refers to a molecule comprising a nucleic acid. For example, the polynucleotide can contain the nucleotide sequence of the full length cDNA sequence, including the 5′ and 3′ untranslated sequences, the coding region, with or without a signal sequence, the secreted protein coding region, and the genomic sequence with or without the accompanying promoter and transcriptional termination sequences, as well as fragments, epitopes, domains, and variants of the nucleic acid sequence. Moreover, as used herein, a “polypeptide” refers to a molecule having the translated amino acid sequence generated from the polynucleotide as defined.

The polynucleotide of the present invention can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons.

The polypeptide of the present invention can be composed of amino acids joined to each other by peptide bonds or modified peptide bonds, i.e., peptide isosteres, and may contain amino acids other than the gene-encoded amino acids. The polypeptides may be modified by either natural process, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural process or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—structure and molecular properties, 2nd Ed., T. E. Creighton, W. H. Freeman and Company, New York (1993); Postranslational covalent modification of proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

An oligonucleotide probe may also be designed to hybridize to the complementary sequence of either the sense or antisense strand of a specific target sequence, and may be used alone or as a pair, such as in DNA amplification reactions, but necessarily will comprise one or more polymorphic loci of the present invention.

As used herein, the terms “nucleotide”, “base” and “nucleic acid” are intended to be equivalent. The terms “nucleotide sequence”, “nucleic acid sequence”, “nucleic acid molecule” and “nucleic acid segment” are intended to be equivalent.

Hybridization probes are oligonucleotides which bind in a base-specific manner to a complementary strand of nucleic acid and are designed to identify the allele at one or more polymorphic loci within the NQO1 gene of the present invention. The probe preferably comprises at least one polymorphic locus occupied by any of the possible variant nucleotides. For comparison purposes, the present invention also encompasses probes that comprise the reference nucleotide at least one polymorphic locus. The nucleotide sequence can correspond to the coding sequence of the allele or to the complement of the coding sequence of the allele, where applicable.

As used herein, the term “primer” refers to a single-stranded oligonucleotide which acts as a point of initiation of template-directed DNA synthesis under appropriate conditions. Such DNA synthesis reactions may be carried out in the traditional method of including all four different nucleoside triphosphates (e.g., in the form of phosphoramidates, for example) corresponding to adenine, guanine, cytosine and thymine or uracil nucleotides, and an agent for polymerization, such as DNA or RNA polymerase or reverse transcriptase in an appropriate buffer and at a suitable temperature. Alternatively, such a DNA synthesis reaction may utilize only a single nucleoside (e.g., for single base-pair extension assays). The appropriate length of a primer depends on the intended use of the primer, but typically ranges from about 10 to about 30 nucleotides. Short primer molecules generally require cooler temperatures to form sufficiently stable hybrid complexes with the template. A primer need not reflect the exact sequence of the template, but must be sufficiently complementary to hybridize with a template. The term “primer site” refers to the area of the target DNA to which a primer hybridizes. The term primer pair refers to a set of primers including a 5′ (upstream) primer that hybridizes with the 5′ end of the DNA sequence to be amplified and a 3′ (downstream) primer that hybridizes with the complement of the 3′ end of the sequence to be amplified.

Representative diseases or malignancies which may be detected, diagnosed, identified, treated, prevented, and/or ameliorated by the SNPs or methods of the present invention include, the following, non-limiting diseases and disorders: breast cancer, lung, bladder, prostatic, ovarian, pancreatic, gastric or colorectal cancer, cancer of the large intestine, non-Hodgkin's lymphoma, head neck cancer, large cell lung carcinoma, small cell lung carcinoma or soft tissue sarcoma or children's tumor or other cancers and malignancies which can be treated with DNA breaking agents such as anthracycline.

With the expression “whether a subject would benefit from being excluded from a therapy” it is meant that subject or patients for whom a certain generally used therapy is ineffective may be identified at an early stage and the subject may be treated with an alternative tailor made therapy adapted to the subject's genotype and response to therapies without having to go through a painful and possible detrimental therapy. In other words the subjects who do not benefit from a treatment or whom a treatment would be detrimental are identified.

Most, if not all human genes occur in a variety of forms which differ in at least minor ways. Heterogeneity in human genes is believed to have arisen, in part, from minor, non-fatal mutations that have occurred in the genome over time. In some instances, differences between alternative forms of a gene are manifested as differences in the amino acid sequence of a protein encoded by the gene. Some minor amino acid sequence differences can alter the stability, reactivity or substrate specificity of the protein. Differences between alternative forms of a gene can also affect the degree the gene is expressed. However, many heterogeneties that occur in human genes appear not to be correlated with any particular phenotype. Known heterogeneties include, e.g. single nucleotide polymorphism (i.e., alternative forms of a gene having a difference at a single nucleotide residue). Other known polymorphic forms include those in which the sequence of larger portions of a gene exhibit numerous sequence differences and those which differ by the presence or absence of portion of a gene.

The present invention provides a novel SNP, which is associated with the response to a certain therapy. The SNPs disclosed herein are useful for diagnosing, screening for, and evaluating the response to a defined therapy in humans. Furthermore, the SNPs and the functionality of their encoded products are useful diagnostic tools.

Particular SNP alleles of the present invention can be associated with an adverse response to a given cancer treatment which is related to lack of normal or functional gene or gene product.

The present invention provides individual SNPs for predicting the response to cancer therapy as well as combinations of SNPs and haplotypes in genetic regions associated with said marker gene. Methods of screening for SNPs useful for selecting a treatment strategy, or excluding the subjects from a treatment are provided. The present invention provides SNPs for identifying a novel association between the presence or absence of predictive marker and response to therapy. The present invention provides novel compositions and methods based on the SNPs disclosed herein, and also provides novel methods of using the known, but previously unassociated, SNPs in methods relating to the response to a therapy. Particular SNP alleles of the present invention can be associated with either a negative response or positive response to a therapy.

Those skilled in the art will readily recognize that polynucleotides may be DNA or RNA. DNA is a nucleic acid molecule, which is a double-stranded molecule. Genes are DNA from a particular site on one strand referring, as well, to the corresponding site on a complementary strand. In defining a SNP position, SNP allele, or nucleotide sequence, reference to an adenine, a thymine (uracil), a cytosine, or a guanine at a particular site on one strand of a nucleic acid molecule also defines the thymine (uracil), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers, may be designed to hybridize to either strand and SNP genotyping methods disclosed herein may generally target either strand. Throughout the specification, in identifying a SNP position, reference is generally made to the protein-encoding strand, only for the purpose of convenience.

References to variant peptides, polypeptides, or proteins of the present invention include peptides, polypeptides, proteins, or fragments thereof, that contain at least one amino acid residue that differs from the corresponding amino acid sequence of the art-known peptide/polypeptide/protein (the art-known protein may be interchangeably referred to as the “wild-type”, “reference”, or “normal” protein). Such variant peptides/polypeptides/proteins can result from a codon change caused by a nonsynonymous nucleotide substitution at a protein-coding SNP position (i.e., a missense mutation) disclosed by the present invention. Variant peptides/polypeptides/proteins of the present invention can also result from a nonsense mutation, i.e. a SNP that creates a premature stop codon, a SNP that generates a read-through mutation by abolishing a stop codon, or due to any SNP disclosed by the present invention that otherwise alters the structure, function/activity, or expression of a protein, such as a SNP in a regulatory region (e.g. a promoter or enhancer) or a SNP that leads to alternative or defective splicing, such as a SNP in an intron or a SNP at an exon/intron boundary. As used herein, the terms “polypeptide”, “peptide”, and “protein” are used interchangeably.

Also other variations, such as alterations, deletions, insertions or replacements of one or more nucleotides, or also epigenetic changes, causing that the subject or the tumor is not capable of producing a normal or functional gene product, can be used for identifying subjects that would benefit from being excluded from cancer therapy. Epigenetic changes for example due to methylation may cause inactivation of the gene, even though the genotype is normal.

A “mutant” gene or gene product and “non-functional” gene or gene product means that a gene of gene product is dysfunctional due to homozygous, hemizygous or other genetic or genomic alterations, such as loss of functional alleles or somatic mutations, or epigenetic changes. A “mutant gene” or “non-functional gene” has undergone mutation or results from change or mutation and means a mutant new genetic character arising or resulting from an instance of mutation, which is a sudden structural change within the DNA of a gene or chromosome of an organism and results in the creation of a new character or trait not found in the wildtype. When a gene or gene product is “mutant or non-functional” it means that “gene or gene product has decreased ability to function. A “mutant or non-functional” gene or gene product may mean that the gene product is lacking.

In the present invention the NQO1 gene carries a change of one or more nucleotides, which results in a non-functional NQO1 gene or gene product. In a preferred embodiment NQO1 gene carries a change in the nucleotide sequence corresponding to the cytosine to thymine substitution at position 609 of the polynucleotide sequence in NCBI sequence ID:J03934.1 or refSNP ID:rs1800566 set forth in SEQ ID NO:4 comprising a c.609C>T allele or NQO1*2 polymorphism, thereby resulting in the amino acid change of proline to serine at position 187, P187S, of the encoded gene product.

A “normal gene product” or “normal functional gene product” or “normal or functional gene product” means a protein or polypeptide encoded by a normal or functional gene and which is characterized by having a fully maintained functionality. In the present invention one functionality is that of the NQO1 protein, which is characterized by an activity which is measurable as described below. The normal form of the NQO1 gene is designated as polymorphic form NQO1*1.

In the present invention the subject is classified to a subset having a mutant or non-functional NQO1 gene if the T allele is present in both copies of the c.609 position, and to a subset having a normal or functional NQO1 gene if one of the alleles present in the c.609 position is C.

The presence or absence of said normal or functional gene and its gene products can be determined by using a multitude of detection methods based on the detection of polynucleotides including DNA or RNA, or proteins or polypeptides in question as demonstrated by in vitro detection of a c.609C>T allele or NQO1*2 polymorphism in the NQO1 gene resulting in the P187S change in a gene product.

A polymorphism in NQO1 is known to result in extremely limited amounts or a total lack of the protein and therefore the detection of the protein or its activity can be used to screen potential subjects. It is known that homozygous carriers of the c.609C>T allele, often referred to as NQO1*2, have no measurable NQO1 protein or protein activity, reflecting very low levels of the NQO1 P187S protein due to its rapid turnover via the ubiquitin proteasomal pathway (Siegel et al., 1999; 2001). Therefore, the genotype of a person may be determined indirectly by detecting the presence or absence of NQO1 protein or its activity. The NQO1 activity may be determined e.g. by using a substrate described in Beall et al., Cancer Res. 54:3196-3201 (1994) and Siegel et al., Mol. Pharmacol., 44:1128-1134 (1993), Siegel et al., Cancer Res., 50:7293-7300 (1990).

The detection of protein and its activity measurement thereby provides a useful method for measuring from a protein containing sample whether the subject would benefit from being excluded from a particular treatment or not. Reduced level or a total lack of the NQO1 protein in a sample can be determined also by methods, such as immunoblotting or immunohistochemistry using a polyclonal or monoclonal antibody specific for NQO1 protein.

The term “lacking a normal functional gene product” means a protein or polypeptide encoded by a gene, which is absent or does not have the function of the normal protein or enzyme as described above. In the present invention it is a mutant gene having one or more SNPs which has the effect that the encoded protein does not have the functionality of normal NQO1 protein or is completely absent. The disappearance of the functionality of NQO1 protein may be caused by a nucleotide variation that may cause the formation of an erroneous mRNA or lead to a rapid destruction by cell.

Presence of NQO1*2 polymorphism (heterozygosity) indicates a lowered response to the therapy in vitro. Presence of two copies of NQO1*2 polymorphism (homozygosity) indicates no response to the therapy or even a detrimental effect of the therapy in vitro as well as among cancer patients.

“Heterozygosity” means that an organism is a heterozygote or is heterozygous at a locus or gene when it has different alleles occupying the gene's position in each of the homologous chromosomes. In other words, it describes an individual that has two different alleles for a trait. In diploid organisms, the two different alleles are inherited from the organism's two parents. For example a heterozygous individual would have the allele combination Pp. In the present invention heterozygosity means e.g. that the presence of a copy of NQO1*2 polymorphism results in reduced NQO1 functionality. In the present invention heterozygosity can be lost (loss of heterozygosity) in tumor cells due to loss of the second allele of c.609C>T and cells become hemizygous for the c.609C>T. In the present invention heterozygous variant (PS) means the allele combination NQO1:NQO1*2.

“Homozygosity” means that an organism is referred to as being homozygous at a specific locus when it carries two identical copies of the gene affecting a given trait on the two corresponding homologous chromosomes (e.g., the genotype is PP or pp when P and p refer to different possible alleles of the same gene). Such a cell or such an organism is called a homozygote. A homozygous dominant genotype occurs when a particular locus has two copies of the dominant allele (e.g. PP). A homozygous recessive genotype occurs when a particular locus has two copies of the recessive allele (e.g. pp). Pure-bred or true breeding organisms are homozygous. For example a homozygous individual could have the allele combinations PP or pp. All homozygous alleles are either allozygous or autozygous. In the present invention homozygous for normal (PP) means that NQO1 locus has the allele combination NQO1: NQO1 is denoted as NQO1*1. In the present invention homozygous for variant (SS) means that functional NQO1 is lacking and is denoted as NQO1*2. In the present invention homozygosity means e.g. the presence of two copies of NQO1*2 polymorphism results in little or no NQO1 functionality.

“Hemizygous” describes a diploid organism which has only one allele of a gene or chromosome segment rather than the usual two. A “hemizygote” refers to a cell or organism whose genome includes only one allele at a given locus. In the present invention hemizygosity means for example that the presence of one copy of NQO1*2 polymorphism results in little or no NQO1 functionality. In the present invention tumor hemizygosity can occur due to loss of heterozygosity (LOH) or inactivation of the other allele or inactivation due to epigenetic mechanisms or due to somatic mutations. Presence of one copy of the c.609C>T allele in the tumor with loss or inactivation of the other allele indicates that the tumor cells are hemizygous for the c.609C>T allele and the subject benefits from being excluded from the treatment.

“Chemotherapy” means the treatment of cancer using specific chemical agents or drugs that are selectively destructive to malignant cells and tissues. It refers primarily to cytotoxic drugs used to treat cancer. In its non-oncological use, the term may also refer to antibiotics (antibacterial chemotherapy). In other words “chemotherapy” means also treatment of disease using chemical agents or drugs that are selectively toxic to the causative agent of the disease, such as a virus or other microorganism. Other uses of “cytostatic chemotherapy agents” are the treatment of autoimmune diseases such as multiple sclerosis and rheumatoid arthritis, the treatment of some chronic viral infections such as Hepatitis, and the suppression of transplant rejections. Broadly, most chemotherapeutic drugs work by impairing mitosis (cell division), effectively targeting fast-dividing cells. As these drugs cause damage to cells they are termed cytotoxic. “Cytostatic chemotherapy agents” are also called “cytostatics”. Some drugs cause cells to undergo apoptosis (so-called “cell suicide”).

As “chemotherapy” affects cell division, tumors with high growth fractions (such as acute myelogenous leukemia and the lymphomas, including Hodgkin's disease) are more sensitive to “chemotherapy”, as a larger proportion of the targeted cells are undergoing cell division at any time. The majority of chemotherapeutic drugs can be divided in to: alkylating agents, antimetabolites, anthracyclines, plant alkaloids, topoisomerase inhibitors. All of these drugs affect cell division or DNA synthesis and function in some way. Some of the cytostatics are phase specific i.e. they inhibit cell division in only certain phase of the cell cycle.

There are a number of strategies in the administration of chemotherapeutic drugs used today. “Chemotherapy” may be given with a curative intent or it may aim to prolong life or to palliate symptoms. Combined modality chemotherapy is the use of drugs with other cancer treatments, such as radiation therapy or surgery. Most cancers are now treated in this way. Combination chemotherapy is a similar practice which involves treating a patient with a number of different drugs simultaneously. The drugs differ in their mechanism and side effects. The biggest advantage is minimizing the chances of resistance developing to any one agent.

“Early curative therapy” comprises a therapy which is given with a curative intent at an early stage of the disease or which is the first therapy given to a subject in need. Early curative therapy comprises modalities that causes DNA breakage and/or triggers apoptotic response. Such modalities comprise chemotherapy, which is carried out with a chemotherapy agent comprising a topoisomerase inhibitor such as topoisomerase inhibitor II.

“Adjuvant chemotherapy” means cancer chemotherapy employed after the primary tumor has been removed by some other method. “Adjuvant chemotherapy” as postoperative treatment can be used when there is little evidence of cancer present, but there is risk of recurrence. “Adjuvant chemotherapy” can help reduce chances of resistance developing if the tumor does develop. It is also useful in killing any cancerous cells which have spread to other parts of the body. This is often effective as the newly growing tumors are fast-dividing, and therefore very susceptible. “Palliative chemotherapy” is given without curative intent, but simply to decrease tumor load and increase life expectancy. For these regimens, a better toxicity profile is generally expected.

Most chemotherapy regimens require that the patient is capable to undergo the treatment. Performance status is often used as a measure to determine whether a patient can receive chemotherapy, or whether dose reduction is required.

“Combination chemotherapy” means that different agents are combined simultaneously in order to enhance their effectiveness. “Induction chemotherapy” means the use of drug therapy as the initial treatment for patients presenting with advanced cancer that cannot be treated by other means. “Neoadjuvant chemotherapy” means the initial use of chemotherapy in patients with localized cancer in order to decrease the tumor burden prior to treatment by other modalities. In other words this preoperative treatment means that initial chemotherapy is aimed for shrinking the primary tumor, thereby rendering local therapy (surgery or radiotherapy) less destructive or more effective. “Regional chemotherapy” means chemotherapy, especially for cancer, administered as a regional perfusion. “Alternative therapy” may be another cytostatic, endocrine agent, treatment or biological treatment indicated for treatment of the specific cancer of the patient.

Topoisomerases are essential enzymes that maintain the topology of DNA. Inhibition of type I or type II topoisomerases interferes with both transcription and replication of DNA by upsetting proper DNA supercoiling. “Topoisomerase inhibitors” are chemotherapy agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle.

“Topoisomerase inhibitors” have become targets for cancer chemotherapy treatments. It is thought that topoisomerase inhibitors block the ligation step of the cell cycle, and that topoisomerase I and II inhibitors interfere with the transcription and replication of DNA by upsetting proper DNA supercoiling. A commonly prescribed class of topoisomerase inhibitors are fluoroquinolones. Examples of topoisomerase I inhibitors include irinotecan and topotecan. Examples of topoisomerase II inhibitors include amsacrine, mitoxantrone, piroxantrone, dactinomycin, anthracyclins, epipodofyllotoxin-derivatives such as etoposide or teniposide, etoposide phosphate.

“Anthracyclins”, which are topoisomerase II inhibitors, also cause breaking of DNA and chromosomal damages, possibly due to the formation of reactive oxidative radicals. Anthracyclins include for example doxorubicin, daunorubicin, idarubicin, aclarubicin or epirubicin. Especially doxorubicin and epirubicin are widely used in chemotherapy since they are broad-spectrum cytostatics.

“Cytostatics” which are used in the “breast cancer treatment” include for example: anthracyclins such as doxorubicin or epirubicin, fluorouracil, methotrexate, mitomycin, mitoxantrone, cyclophosphamide, taxans such as docetaxel or paclitaxel, vinca-alcaloids such as vincristine, vindecin or vinorelbine. The most common combinations of cytostatics include for example CMF and CAF/FEC (cyclophosphamide+doxorubicin/epirubicin+5-fluorouracil).

“p53”, also known as tumor protein 53, is a transcription factor that regulates the cell cycle and hence functions as a tumor suppressor. The p53 protein normally plays a central role in the cellular response to a variety of different stresses, particularly stresses arising from DNA damage caused by radiation, oxidative stress or other agents: once activated by a stress, p53 either induces cell-cycle arrest (termination of cellular proliferation) or facilitates programmed cell death (apoptosis) (Kastan 2007). The term “p53-defective” means that the gene coding for a p53 is not functional or is imperfect or has a defect or the whole gene is lacking. In other words “p53-defective” means the failure of an organism to develop properly p53.

The term “immunopositive” means that the sample is positive in immunohistochemistry. Immunohistochemistry is the process of localizing proteins in cells of a tissue section exploiting the principle of antibodies binding specifically to antigens in biological tissues and is used to understand the distribution and localization of biomarkers in different parts of a tissue. Immunohistochemical staining is widely used in the diagnosis and treatment of cancer. Specific molecular markers are characteristic of particular cancer types. In the present invention “p53-immunopositive” sample has been detected with a p53 antibody in immunohistochemistry and refers to positive result in immunohistochemistry. “p53 immunopositivity” means defected p53. Mutated p53 is not degraded as it is meant to be and this results in p53 immunopositivity. In other words defected gene product is accumulated in the cells and can be detected by immunohistochemical analysis. The term “immunonegative” means that the sample is negative in immunohistochemistry. The term “p53 immunonegative” means that a sample is negative or has a very low expression when detected with a p53 antibody. p53 is broken down rapidly and is not accumulated meaning that it can not be readily detected by immunohistochemistry.

“p53 immunopositive heterozygous” means that a subject heterozygous for the c.609C>T allele or polymorphism of NQO1 gene has a defected p53 and is detected immunopositive in immunohistochemical analysis.

The expression that the method can be used to selecting a cancer therapy for treatment of metastatic cancer means that the subject suffering form a cancer of malignancy is detected with metastasis and the method of the present invention is used to determine the beneficial cancer therapy. The subject may have been treated with any cancer therapy to cure a primary tumor. The genotyping of determining the presence of a mutant or non-functional NQO1 gene or gene product, or absence of a normal or functional NQO1 gene or product from a sample of the subject comprising healthy or tumor cells is carried out. The determination is done before the onset of chemotherapy to determine whether the subject would benefit from the intended therapy such as anthracyclin based chemotherapy. The time frame between the treatments may vary up to several years.

### GENERAL DESCRIPTION OF THE INVENTION

The present invention is based on the surprising finding that it is possible based on the presence of a mutant or non-functional or absence of a normal or wild type gene or a functional gene encoding NQO1 gene product to determine whether a subject would benefit from being excluded from a treatment. In other words the invention relates to the finding that a decrease or lack of NQO1 gene product or deficiency of NQO1 gene predicts poor survival after therapy. The method of the present invention comprises detecting from a sample of the subject the presence of a mutant or non-functional or absence of a normal or functional NQO1 gene or gene product or a specific polymorphic variant of NQO1 gene or gene product. The detection may comprise any sequence specific genotyping method or phenotyping method or any method based on DNA, RNA or amino acid. The precise detection method is not critical as long as the method is capable of differentiating that the functional gene or gene product is lacking.

The absence of the normal or functional NAD(P)H:Quinone oxidoreductase 1 (NQO1) is due to the fact that the subject or the tumor lacks a functional NQO1 gene or gene product and/or that the subject or the tumor is not capable of producing a normal or functional NQO1 gene product.

The present invention provides a significant improvement for classifying cancer subjects which would benefit from being excluded from the normally applied cancer therapy and would benefit from being directly treated with an alternative treatment regimen. The invention is particularly useful for identifying subjects who carry the NQO1*2 genotype and would benefit from being excluded from anthracyclin treatment. NQO1 polymorphism affects the level of NQO1 protein expression so that NQO1*2 homozygous subjects are not able to produce stable NQO1 protein. The method is particularly useful for identifying NQO1*2 heterozygous subjects suffering from a cancer comprising a p53 immunopositive tumor and who would benefit from being excluded from cancer therapy.

The method of the invention especially enables the determination by genotyping before the onset of the chemotherapy, especially anthracyclin based chemotherapy, whether the patient would benefit from said therapy. The patients with the NQO1 gene variation do not benefit from the said treatment and their condition may even be impaired. Said NQO1 polymorphism can be detected from both the healthy and tumor cells of the patient. The results of the genotyping can be utilized in the treatment of recurred cancer or malignancy, metastatic cancer or newly detected primary cancer of malignancy. The genotyping can be done even if the subject does not yet suffer from a cancer or malignancy. The NQO1 genotyping carried out in subject's healthy cells indicates whether a healthy cell or tumor cell is able to produce a functional NQO1 protein at any stage of a possible cancer treatment of during the progression of a cancer or malignancy.

An example is a test kit comprising at least one substrate reagent for detecting NQO1 functionality or at least one antibody to detect presence or absence of the NQO1 gene product in a sample from a subject, e.g. the presence or absence of the enzyme NQO1 or the activity of the enzyme NQO1 in a sample representative of the subject's inherited genotype, or the genotype of the tumor. The present invention could be utilized in a diagnostic tool for determining whether a subject would benefit from being excluded from a treatment and comprising at least one polynucleotide which is capable of recognizing the presence of a mutant or non-functional gene or gene product of NQO1 gene, or absence of a normal or functional gene or gene product of NQO1 gene from a sample of the subject. The polynucleotide is complementary to a sequence encoding NQO1 or a fragment thereof. The tool also comprises compatible auxiliary reagents and devices, including reagents, labels, buffers, reference samples, amplification means, sequencing means, detergents, biochemical regents, detection means and devices including a solid support such as membrane, filter, slide, plate, chip, dish or microwell composed of material selected from the group consisting of glass, plastics, nitrocellulose, nylon, polyacrylic acids and silicons and instructions for use. Alternatively, said diagnostic tool comprises at least one substrate reagent for detecting NQO1 functionality in a sample or at least one antibody specific for NQO1 gene product in a sample and compatible auxiliary reagents and devices, wherein a result presenting the absence of said normal or functional gene or gene product indicates that the subject would benefit from being excluded from a treatment.

Another example is a predictive marker composition useful in the method of the present invention comprising at least one polynucleotide which is capable of recognizing the presence of a mutant or non-functional gene or gene product of NQO1 gene, or absence of a normal or functional gene or gene product of NQO1 gene from a sample of the subject. The polynucleotide is complementary to a sequence encoding NQO1 or a fragment thereof. The composition also comprises compatible auxiliary reagents and devices. Alternatively, said diagnostic tool comprises at least one substrate reagent for detecting NQO1 functionality in a sample or at least one antibody specific for NQO1 gene product in a sample and compatible auxiliary reagents and devices. Said predictive marker composition is useful in determining whether a subject would benefit from being excluded from a treatment.

Another example is the use of a polynucleotide sequence encoding NQO1 gene or fragments thereof or a substrate reagent or antibody specific for NQO1 gene product in detection of the presence of a mutant or non-functional or absence of a normal or functional gene or gene product, wherein the presence of a mutant or non-functional gene or a gene product or absence of a normal or functional gene or gene product indicates that the subject would benefit from being excluded from said cancer treatment.

Another example is a marker composition for determining whether a subject would benefit being excluded from a treatment in accordance with the method, wherein the composition comprises at least one polynucleotide for detecting the presence of a mutant or non-functional or absence of a normal or functional NQO1 gene or at least one substrate reagent or antibody detecting a gene product of NQO1 gene from a sample of the subject, wherein the polynucleotide is complementary to a sequence encoding NQO1 or a fragment thereof, or the substrate reagent or antibody specific for a gene product of NQO1 gene and compatible auxiliary reagents and devices.

### The Preferred Embodiment Related to the Use of the NQO1 and its Gene Products

The present invention discloses for the first time the NQO1*2 genotype as a prognostic and predictive factor for selecting a treatment, preferably cancer therapy, more preferably breast cancer treatment. The present invention is based on the surprising finding that it is possible based on the presence of a mutant or non-functional NQO1 gene or gene product, or absence of a normal or functional NQO1 gene or gene product to determine, whether a subject would benefit from being excluded from a given cancer therapy. Especially it has been shown that homozygous cytosine to thymine substitution at position 609 in the polynucleotide sequence NCBI sequence ID:J03934.1, ref SNP IDS:rs1800566, named also c.609C>T allele or NQO1*2 polymorphism, resulting in the change of proline to serine (P187S) in an encoded gene product, is associated with poor survival among cancer patients, preferably breast cancer patients, especially after anthracycline-based adjuvant chemotherapy with epirubicin (FEC). The method for selecting a cancer therapy based on subject's genetic background enables to classify subjects in at least two subsets wherein one subset having a normal or functional NQO1 gene or gene product may be treated with cancer therapy and another subset having a mutant or non-functional NQO1 gene or gene product would benefit from being excluded from said cancer therapy. The method of the invention enables the determination by genotyping before the onset of the chemotherapy, especially anthracyclin based chemotherapy, whether the patient would benefit from said therapy. The patients with the NQO1 gene variation do not benefit from the said treatment and their condition may even be impaired.

An association between homozygous NQO1*2 and poor survival among breast cancer patients, especially after anthracycline-based adjuvant chemotherapy with epirubicin was shown. NQO1*2 homozygosity, combined with epirubicin treatment and p53 immunopositive tumors, was identified as an independent, highly significant predictor of poor outcome.

Today, there are no accepted factors predictive for chemotherapy resistance in breast cancer. To optimize performance of a treatment, preferably an adjuvant chemotherapy, novel predictive factors are required that would help to select the best treatment regimen for individual patients. The present invention identifies such a useful predictive marker, the genetic variant NQO1*2 to be used in a screening method for determining whether a subject would benefit from being excluded from a treatment. A highly significant association between NQO1*2 homozygosity and adverse breast cancer outcome as well as higher metastatic potential was detected.

Genetic and clinical observations are functionally validated and are mechanistically supported by in vitro studies where response to epirubicin was. Consistently, NQO1-deficient NQO1*2 cells (SS) were more resistant to epirubicin than the NQO1-proficient cells (NQO1*1), and enhanced levels of NQO1 rendered cells more sensitive to epirubicin treatment.

Taken together, the clinical and functional findings suggested reduced epirubicin and cytotoxicity in NQO1*2 homozygous breast cancer, with a drastic reduction in survival among patients who have undergone treatment, preferably adjuvant—particularly epirubicin-based—chemotherapy. Among such patients, NQO1 genotype provides a predictive factor for treatment. The NQO1 status may be used to provide predictive information also for other types of malignancies. In the present invention a NAD(P)H:Quinone oxidoreductase 1 (NQO1) gene, which carries a c.609C>T allele resulting a protein encoding P187S is used as the predictive marker. In a preferred embodiment of the present invention the method comprises the detection of the presence of a mutant or absence of normal or functional gene or gene product, including transcription or translation products. The invention is based on genotyping and phenotyping methods, applying techniques based on specific measurement of DNA, RNA or amino acid sequences or functionality. Examples of such sequence specific genotyping methods include but are not limited to a technique for single nucleotide polymorphism (SNP) detection and genotyping, such as restriction fragment length polymorphism PCR (RFLP-PCR), SSCP, allele specific hybridization, primer extension, allele specific oligonucleotide ligation or sequencing. The so called minisequencing method described in WO 91/13075 applying DNA polymerase for identifying SNPs may be used as well as methods applying reverse transcriptase for identifying SNPs.

The malignancy or cancer may be selected from breast cancer, lung, bladder, prostatic, ovarian, pancreatic, gastric or colorectal cancer, cancer of the large intestine, non-Hodgkin's lymphoma, head neck cancer, large cell lung carcinoma, small cell lung carcinoma or soft tissue sarcoma or children's tumor. Preferably, the cancer is breast cancer. The present method is useful in connection with above mentioned cancers and malignancies, DNA breaking agents, such as anthracyclin-based adjuvant chemotherapy is also used in the treatment of these cancers and malignancies.

The sample may be substantially any sample. The sample type is not critical as long as it represents the subject's inherited genotype, or genotype in the tumor. The sample may be obtained from any cell. The samples may be tumor cells or tissues or fluids, which contain nucleic acids or proteins or polypeptides, polynucleotide, or transcript. Such samples include, tissue isolated from the subject to be treated and tissues such as biopsy and autopsy samples, or comprise frozen sections taken for histological purposes, archival samples, blood, plasma, serum, sputum, stool, tears, mucus, hair, skin, etc. The samples also include explants and primary and/or transformed cell cultures derived from patient tissues.

In an embodiment of the invention the treatment comprises a modality or therapy that causes DNA breakage and/or triggers apoptotic response, more preferably the modality that causes DNA breakage and/or triggers apoptotic response is chemotherapy. Preferably chemotherapy is carried out with a chemotherapy agent comprising topoisomerase II inhibitor or derivatives thereof, or any agent causing DNA breakage or derivatives thereof. Examples of such chemotherapy agents include but are not limited to topoisomerase II inhibitor comprising amsacrine, mitoxantrone, piroxantrone, dactinomycin, anthracyclins, epipodofyllotoxin-derivative such as etoposide, teniposide, or etoposide phosphate. Examples of anthracyclins include but are not limited to comprise doxorubicin, daunorubicin, idarubicin, aclarubicin or epirubicin. Most preferably the treatment comprises anthracycline-based adjuvant chemotherapy with epirubicin.

There is great need for novel predictive factors that would help to predict the response to a therapy and to select the best treatment regimen for individual patients. The present invention accordingly relates to cancer treatment, particularly a method for selecting of the best treatment regimen for an individual patient. To optimize performance of a treatment, preferably an adjuvant chemotherapy, novel predictive factors are required that would help to select the best treatment regimen for individual patients.

Having now generally described the invention, the same will be more readily described through reference to the following examples, which are provided by way of illustration and are not intended to be limiting of the present invention.

### Example 1

### Materials and Methods

### Patients and Controls

The germline NQO1 codon 187 genotype c.609C>T was defined among an extensive series of 883 Finnish familial breast cancer patients, two independent sets of unselected breast cancer patients of 884 and 886 patients, and a set of 698 geographically matched healthy female population controls. The unselected series are representative of the patients diagnosed with breast cancer during the collection period.

The familial series, collected at the Helsinki University Central Hospital as previously described (Eerola et al., 2000) includes a total of 883 patients with invasive breast cancer. 389 of them had a stronger family history (three or more first or second degree relatives with breast or ovarian cancer in the family, including the proband), as verified through the Finnish Cancer Registry and hospital records, whereas 494 unrelated breast cancer cases reported only a single affected first-degree relative. BRCA1 and BRCA2 mutations had been excluded in all of the high-risk families, as well as in 306 (61.9%) of the two case families, by screening of the entire coding regions and exon-intron boundaries using protein truncation test (PTT) and denaturing gradient gel electrophoresis (DGGE), or as previously described (Vahteristo et al., 2001).

The first series of 884 unselected breast cancer patients studied were collected at the Department of Oncology, Helsinki University Central Hospital in 1997-1998 and 2000 and cover 79% of all consecutive, newly diagnosed breast cancer cases during the collection periods (Kilpivaara et al., 2005; Syrjakoski et al., 2000). A total of 40 of these unselected patients had non-invasive breast cancer and were excluded from these analyses.

The second unselected series, containing 886 consecutive newly diagnosed patients with invasive breast cancer, unselected for family history, were collected at the Helsinki University Central Hospital 31 Oct. 2001-29 Feb. 2004 and covers 87% of all such patients treated at the Department of Surgery during the collection period. Histopathological data was collected from pathology reports for all the primary invasive breast tumors, including contralateral tumors, available among the patients in the two unselected sample sets (n=1757) as well as the familial set (n=1045). The data set in this study includes information on tumor histology, grade, estrogen receptor (ER) and progesterone receptor (PgR) status, p53 immunohistochemical expression and tumor diameter (T), nodal status (N) and distant metastases (M). The p53 immunohistochemical expression data was obtained either from pathology reports or, when available, studied by immunohistochemical staining of tumor tissue microarrays (TMA) as previously described (Tommiska et al., 2005). p53 immunopositivity (staining levels >20% of cells were scored as positive) was determined by two pathologists who independently reached virtually identical scores. TMA data was obtained from 664 of the familial tumors and 571 of the unselected tumors, covering 87% and 66% of all p53 expression data in the material, respectively. Information on adjuvant chemotherapy, radiotherapy and endocrine treatment was collected from patient records.

The data set also includes the age at the time of (first) breast cancer diagnosis and overall survival (in days). The duration of follow-up ranged from 32 to 2958 days (median: 1860; mean: 1778; SD: 505). Age at the time at diagnosis ranged from 22 to 96 years (median: 55.5; mean: 56.6; SD: 12.0). Allele and genotype frequencies in the normal population were determined in 698 healthy female population controls collected from the same geographical region.

### Genotyping

The genotyping of DNA samples from the first set of unselected patients as well as the population controls was performed using Amplifluor™ fluorescent genotyping (K-Biosciences, Cambridge, UK, http://www.kbioscience.co.uk). The samples that failed to produce unambiguous allele calls in the first analysis were re-genotyped with the RFLP assay described below. For quality control, a total of 228 samples (8.9% of all cases) were genotyped using both genotyping methods with 100% (228 out of 228) concordance between duplicates.

The second unselected set and the familial set were genotyped with a restriction fragment length polymorphism (RFLP) assay. For the NQO1 c.609C>T RFLP assay, we designed a 279 by PCR amplicon containing one HinfI restriction site specific to the NQO1*2 allele. After digestion according to the enzyme manufacturer's instructions (New England BioLabs, Beverly, Mass., USA; http://www.neb.com/), PCR product containing the NQO1*2 allele was cleaved into fragments of 152 and 127 base pairs, readily distinguishable on regular 2% agarose gels, whereas wild type amplicons remain intact. The primers used to produce the amplicon were 5′-CCT GAG GCC TCC TTA TCA GA-3′ (forward) (SEQ ID NO:1) and 5′-AGG CTG CTT GGA GCA AAA TA-3′ (reverse) (SEQ ID NO:2).

### Statistical Analysis

The clinical and biological variables were tested for association by univariate analysis. Independent variables were compared with the chi-square test. Univariate analyses of survival were performed by calculating Kaplan-Meier survival curves and comparing subsets of patients using log-rank and Breslow tests. Only incident cases (less than 6 months between diagnosis and sample collection) were included in the survival analyses. In order to characterize the relationship between NQO1 genotype and prognosis, survival analysis was carried out in subgroups of cases based on histopathological characteristics (p53 immunopositivity, axillary node metastasis, hormone receptor status), and types of anticancer treatment, in addition to the whole unselected set of patients. In addition to patient-specific overall survival, tumor-specific Kaplan-Meier analyses of time-to-metastasis, time-to-relapse and generic disease-free survival (time to either metastasis, relapse or a new primary cancer) were performed using the parameters described above. These survival analyses were carried out among the familial and first unselected series, as they had sufficient follow-up times for survival analysis. To exclude survival bias in the study material, only incident cases (less than six months between diagnosis and sampling) were used in the survival analyses. For bilateral cases, follow-up was assigned to start from the first primary invasive breast carcinoma, and continued until a fatal event or the end of follow-up; the second tumor was ignored. All p-values are two-sided and p-value<0.05 was considered significant. The data were analyzed using SPSS for Windows v12.0.1 (SPSS Inc., Chicago, Ill., USA). The sample set eligible for survival analyses is described in detail in Table 3.

To explore the effects of several variables and their interaction terms on survival, a Cox's proportional hazards regression model was constructed using a stepwise method, as implemented in the Forward Conditional algorithm of SPSS v12. Briefly, the algorithm attempts to pick the best combination of prognostic factors to explain the mortality in the study population. As a starting point, the algorithm starts with a pool of available variables, but zero covariates in the model. At each step, the algorithm adds a covariate from the pool of available variables, or removes an existing covariate from the model, based on which stepwise change improves the model the most. This is repeated until the algorithm arrives at a combination of covariates where no statistically significant improvement to the model can be achieved via any stepwise change. Hazard ratios are provided for each covariate.

To evaluate the independence and proportional hazard ratio of NQO1*2 homozygosity among prognostic factors in breast cancer, a Cox's proportional hazards model was generated without any interaction terms. Additionally, two proportional hazards models with interaction terms were constructed: one was based on clinicopathological factors alone, while the other included information on the types of anticancer treatment administered to the patients. The variables and interaction terms included in these analyses are described in Table 4.

### Cell Culture

The cell lines used in the experiments included p53 wildtype (wt) immortalized B-cell lymphoblasts from patients (NQO1 001 (PP), NQO1 003 (PS) and LBL51 (SS), the p53 wt breast cancer cell lines MCF7neo6 (PS), MCF7DT9 (PS but genetically modified to overexpress NQO1 (Siemankovski et al. 2000), p53 mutant MDA MB-157 (PP) and MDA MB-231 (SS), as well as dominant negative p53 (p53DD) expressing U2OS osteosarcoma cells. All cell lines were maintained at 37° C. under a humidified atmosphere at 5% CO₂. All reagents used for cell culture were obtained from GIBCO (Gibco Invitrogen Cell Culture, USA). MCF7 neo6 and DT9 breast cancer cells were kindly provided by M. Briehl and cultivated as previously described (Siemankowski et al., 2000). The B-cell lymphoblast cell lines derived from patients were immortalized with Epstein-Barr virus transformation. Cell lines were cultivated in RPMI supplemented with 10% serum, 100 U Penicillin and 100 μg/ml Streptomycin. Dominant negative p53 (p53DD) expressing U2OS osteosarcoma cells (Mailand et al., 2000) were cultivated in DMEM supplemented with 10% serum, 100 U Penicillin and 100 μg/ml Streptomycin, G418, Puromycine and Tetracycline. MDA MB-157 and MDA MB-231 breast cancer cells were cultivated in DMEM supplemented with 10% serum, 100 U Penicillin and 100 μg/ml Streptomycin.

### Plasmids

The plasmids used were pEFIRES-NQO1 encoding wild type human NQO1 (EFNQ13, MDA MB-231-NQO1) and pEFIRES-empty for vector controls (EFI6, MDA MB-231-empty), pS UPER-NQO1 expressing NQO1 shRNA (NQ12) and pSUPER-empty (ZEO6) [obtained from Gad Asher, Weizmann Institute of Science, Israel (Asher et al., 2005].

### Transfections

1.5E6 cells were seeded in a 10 cm dish one day before transfection. Transfections were carried out using FuGENE 6 (Roche, Switzerland) according to the manufacturer's protocol. 24 h after transfection cells were transferred to fresh dishes in different concentrations low enough to allow growth of single cell clones and selection reagent Zeocin was applied. Clones were picked 12 days later and analyzed.

### Epirubicin, Methotrexate and TNF Treatment

Epirubicin was obtained from Pharmacia (Farmorubicin, Pharmacia Corporation, Chicago, Ill., USA). Aqueous stock solution with a concentration of 2 mg/kg was kept light shielded at 4° C. and was diluted to the appropriate concentrations in culture medium right before treatment of the cells. Methotrexate (MTX, Sigma Chemicals) was dissolved in mildly alkalized PBS and kept frozen in a stock concentration of 10 mM. hTNFα (Roche Applied Science, Indianapolis, Ind., USA) was diluted in appropriate medium right before use. Cycloheximide in a final concentration of 1 μM was added to all cells (except MCF7) 3 h prior to TNF treatment.

### Cell Proliferation and Viability

The effects of Epirubicin and TNF on cell survival were analyzed using proliferation and viability assays. Proliferative activity was assessed by the MTT-like AlamarBlue assay according to the manufacturer's protocol (BioSource International, Camarillo, Calif.). Cells were homogenously seeded in 96 well plates and treated with increasing concentrations of Epirubicin 24 h later. At the indicated timepoints Alamar Blue was added and 4 h later absorption was measured at 570 and 630 nm using a Versamax spectrophotometer. Every treatment was performed in triplicates and each experiment was at least repeated twice. Cellular viability was determined by collecting detached and adherent cells at the indicated timepoints after Epirubicin treatment. Cells were harvested by centrifugation and resuspended in the corresponding medium. Dead cells were stained with SYTOX green (Cambrex, USA) while the overall amount of cells was assessed by Hoechst staining Viability was determined by counting % SYTOX positive cells by fluorescence microscopy. Experiments were performed in duplicate and repeated once.

### Cellular Lysates and Western Blotting

Floating and attached cells were collected at the indicated timepoints after treatment, washed once with PBS and lysed with lysis buffer (Lukas et al., 1998). Cellular lysates were analyzed by immunoblotting using the antibodies for p53, p21, NQO1 (all from Santa Cruz Biotechnology, Inc., Santa Cruz, Calif., USA), PARP (BD Biosciences PharMingen, San Diego, Calif., USA), α-tubulin (Sigma, Sigma-Aldrich, St. Louis, Mo., USA), Mcm7 (DCS-141) and the phospho-specific antibodies for Ser15-p53 (Cell Signaling) and Ser139-γ-H2AX (Upstate). Cellular lysates were obtained from three independent experiments one representative immunoblot is shown.

### Immunofluorescence and Immunohistochemistry

Nuclear translocation of NF-κB/p65 subunit was detected using a rabbit NF-κB/p65 antibody (Santa Cruz Biotechnology, Inc., Santa Cruz, Calif., USA). Tissue staining for NF-κB was performed using a rabbit monoclonal antibody (Abcam, Cambridge, UK). See Codorny-Servat et al. (2006) and Jenkins et al. (2007) for details on the immunostaining protocols.

### Example 2

### NQO1*2 Genotype is not Associated with Breast Cancer Risk

NQO1 genotypes were defined in 2534 breast cancer patients and in 698 healthy controls. The average genotype frequencies in the breast cancer patient series and population controls were 66.7% NQO1*1 (PP), 30.3% heterozygous variant (PS) and 3.0% NQO1*2 (SS). The genotype and allele frequencies were similar among the population controls and breast cancer patients, as well as in patient subgroups stratified by family history of breast cancer or age of diagnosis (Table 5). Oral contraceptive use of the patients did not modulate breast cancer risk by NQO1*2 (genotype frequencies 68.2% (PP), 28.6% (PS), 3.2% (SS) among 770 patients with OC use vs. 66.0% (PP), 30.3% (PS), 3.7% (SS) among 673 patients who never used oral contraceptives) and neither did hormone replacement therapy. No association of the different genotypes with any of the histopathological parameters was observed, aside from p53 immunopositivity (suggestive of p53 mutation) being more common among NQO1*2 homozygotes with a nominally significant p-value (Table 1).

### Example 3

### NQO1 Genotype Impacts Breast Cancer Survival

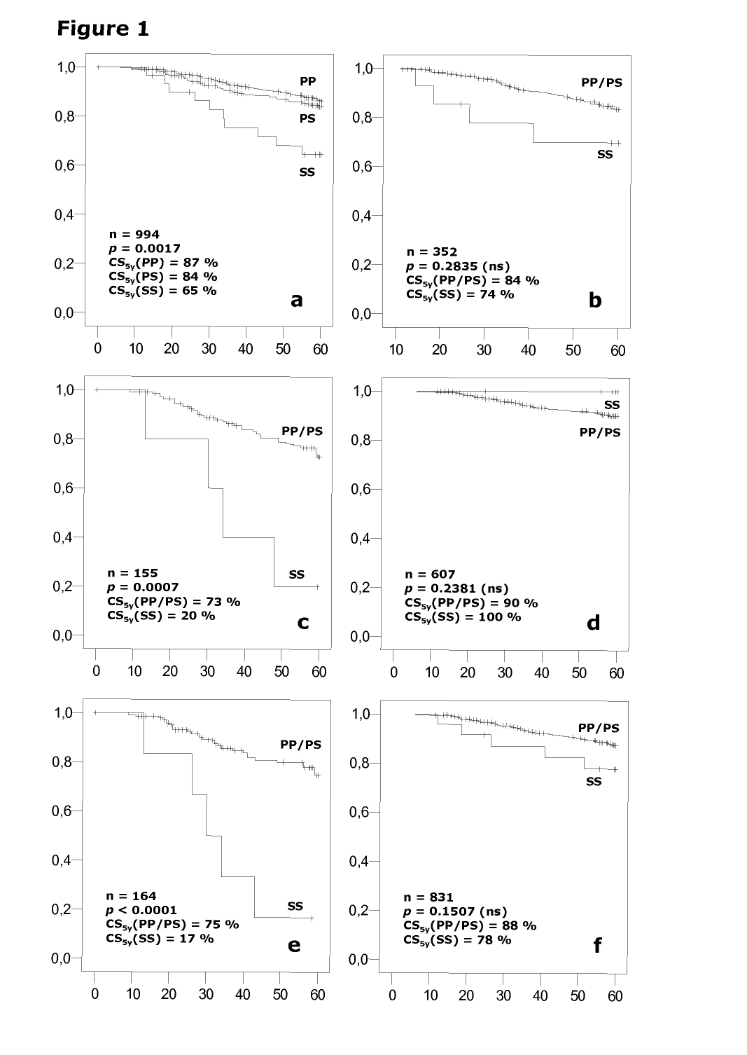

Kaplan-Meier survival analysis showed that NQO1*2 homozygous breast cancer patients had poorer survival than patients with other genotypes, with a five-year cumulative survival (CS_{5y}) of 65% vs. 87% among other genotypes (p=0.0017) (FIG. 1a). The survival curve of the heterozygous patients resembled that of wild-type homozygotes. Subgroup analyses revealed that the NQO1*2-genotype-associated reduced survival was highly evident among patients with positive p53 immunohistochemistry (CS_{5y} 20% SS vs. 73% PP/PS, p=0.001), whereas among p53-low cases NQO1 genotype did not affect survival (FIG. 1c, d). A similar effect was seen among patients who had received adjuvant chemotherapy (CS_{5y} 40% SS vs. 81% PP/PS, p=0.001) but not among the non-treated group, nor among the endocrine therapy group (FIG. 1b, f; see also Table 6). Estrogen and progesterone receptor status did not modulate the impact of NQO1*2 homozygosity on patients' outcome (data not shown).

When the type of adjuvant chemotherapy was factored in, NQO1*2 homozygosity had the most dramatic impact on survival among the FEC (5-fluorouracil (5-FU), epirubicin, cyclophosphamide) treated group (CS_{5y} 17% SS vs. 75% PP/PS, p<0.0001) (FIG. 1e). No such effect was observed among patients treated by non-anthracyclines, especially CMF (cyclophosphamide, methotrexate, 5-FU; CS_{5y} 75% (SS) vs. 86% (PP/PS), p=0.5691, n=193), although this cannot be excluded with statistical methods alone (95% C.I. 33%-100% for the SS homozygotes). The five-year cumulative survival data for all subgroups are shown in Table 6. Consistent with overall survival, NQO1*2 homozygosity was also associated with shorter metastasis-free survival in the same subgroups (Table 6).

### Example 4

### FEC-Treated NQO1*2 Homozygous Patients have Poor Prognosis

In the multivariate Cox's proportional hazards analysis, the interactions between NQO1*2 genotype, positive p53 immunohistochemistry and FEC-treatment emerged as highly significant independent prognostic factors (Table 2). The risk ratio of the interaction between NQO1*2 homozygosity and p53 immunostaining was comparable to that of tumor size (T), lymph node metastasis (N) and distant metastasis (M), even after correcting for the independent prognostic value of p53 immunostaining, while the interaction between NQO1*2 homozygosity and FEC treatment (p<0.001, R.R. 12.69) contributed considerably more to the overall hazard than any other factor. Interestingly, when p53 status was factored in an even higher prognostic value (R.R. 13.61, 95% C.I. 3.86-47.94, p<0.001) was observed. This suggests that the interactions between NQO1*2 homozygosity and p53 immunopositivity on one hand and NQO1*2 homozygosity and FEC treatment on the other are part of one mechanism that affects breast cancer survival in NQO1*2 patients.

### Example 5

### NQO1 Enhances Sensitivity to Epirubicin in Cultured Human Cells

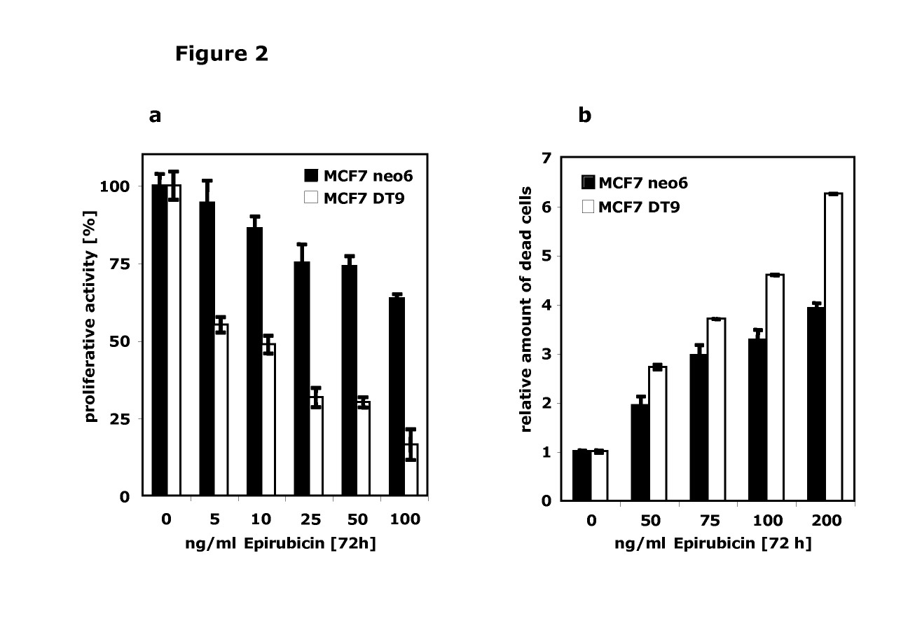

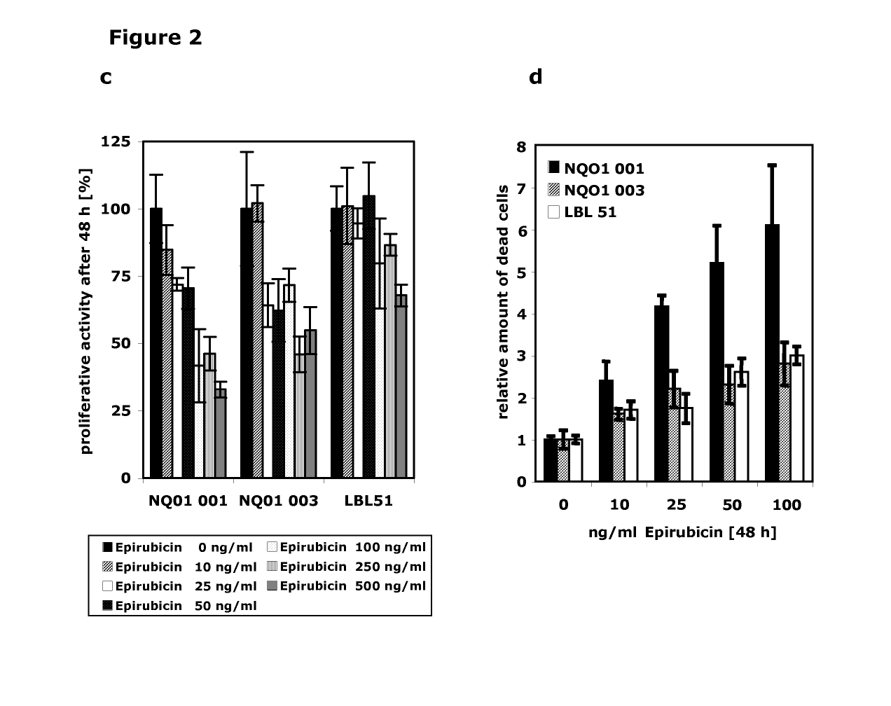

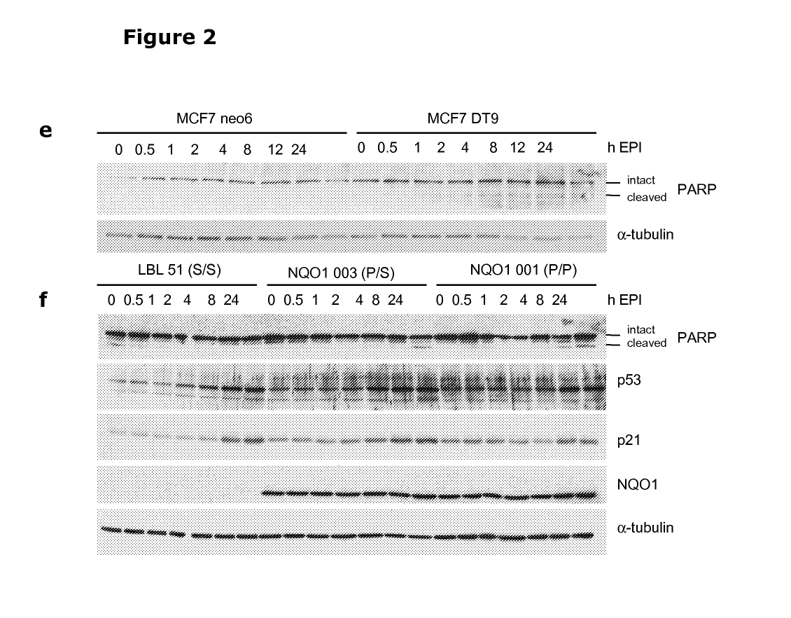

Given that survival after epirubicin-based adjuvant chemotherapy was strongly influenced by NQO1 status, we analyzed epirubicin-induced cell death and the involved pathways in vitro. The p53-wildtype, NQO1-heterozygous (PS) breast cancer cell line MCF7 was stably transfected with NQO1 resulting in the NQO1 overexpressing cell line MCF7DT9 with much greater NQO1-activity than the vector control cell line MCF7neo6 (Siemankowski et al., 2000). NQO1 overexpression increased the sensitivity to epirubicin treatment as shown by the dose-dependent reduction of proliferative activity (FIG. 2a). Consistent with reduced proliferation, cell viability of MCF7DT9 cells was markedly lower after treatment with epirubicin compared to control MCF7neo6 cells (FIG. 2b).

Next, we analyzed the response to epirubicin in EBV-immortalized B-cell lymphoblastoid cell lines established from breast cancer patients with different NQO1 genotype. Proliferative activity was reduced with increasing concentrations of epirubicin measured after 48 h of treatment (FIG. 2c). Homozygous NQO1*2 (SS) lymphoblasts (LBL51) were less sensitive to epirubicin than the homozygous NQO1*1 (PP) cells (NQO1 001), while the heterozygous PS cells (NQO1 003) showed intermediate sensitivity. Correspondingly, the amount of dead cells after 48-hour treatment was higher in the homozygous PP NQO1-proficient cells than in either heterozygous PS or homozygous SS cells (FIG. 2d).

Epirubicin-induced cell death was further monitored by immunoblotting analysis of Poly(ADP-ribose) Polymerase (PARP)-cleavage in both MCF7 (FIG. 2e) and lymphoblast cell extracts (FIG. 20. PARP-cleavage was most evident in the cell lines with higher NQO1 levels (MCF7DT9 and NQO1 001) and absent in cells with undetectable NQO1 (LBL51), supporting our findings from the viability assays.

### Example 6

### Transient Defect of the p53/p21 Pathway in NQO1*2 (SS) Cells

NQO1 protects the tumor suppressor protein p53 against ubiquitin-independent degradation via the 20S proteasome (Asher et al., 2001; 2002a; 2002b). Consistent with these findings, p53 levels in untreated NQO1*1 lymphoblasts (NQO1 001) were higher than in cells from NQO1-heterozygous or SS homozygous patients (FIG. 2f). Furthermore, p21, a transcriptional target of p53, was initially more abundant in NQO1*1 cells, suggesting overall higher p53 transcriptional activity in NQO1-normal cells. In response to epirubicin, p53 abundance increased and by 24 h of treatment reached similar levels in all three cell lines (FIG. 2f), likely reflecting the NQO1-independent stabilization of p53 due to uncoupling of mdm2 from p53 after DNA damage (Lavin et al., 2006).

### Example 7

### Role of p53 in NQO1-Mediated Cell Death Induced by Epirubicin but not by Tumor Necrosis Factor-α (TNF)

The detectable yet not dramatic contribution of NQO1 to p53 stabilization indicated that NQO1 deficiency likely contributes to the overall survival effects by additional mechanism(s). Given that MCF7DT9 cells overexpressing NQO1 are more sensitive to TNF than MCF7neo6 cells (Siemankowski et al., 2000), and that breast cancer patients have elevated plasma levels of TNF (Perik et al., 2006), we argued that response to TNF could represent such a clinically relevant additional pathway.

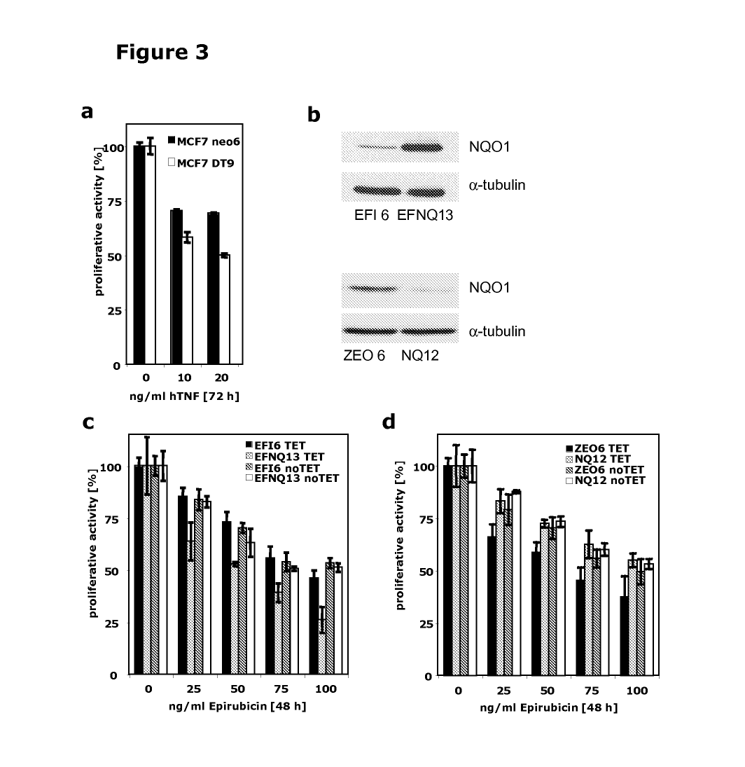

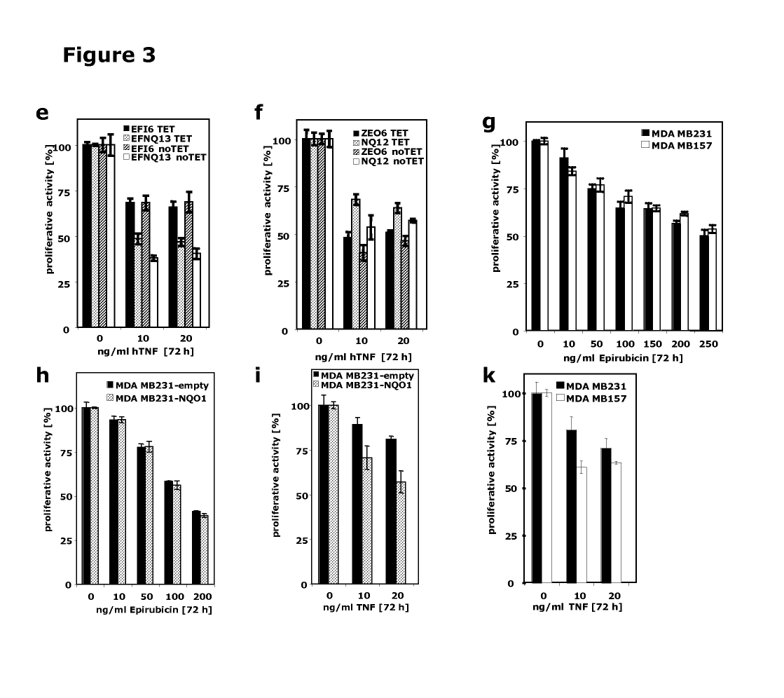

To clarify the roles of p53 and NQO1 in epirubicin-versus TNF-induced, NQO1-mediated cell death, p53DD-U2OS cells (NQO1*1, PP) containing a tetracycline-repressible expression of a dominant-negative mutant of p53 (p53DD) were transfected with pEFIRES-NQO1 to overexpress NQO1 (EFNQ13) or with pSUPER-NQO1 to knockdown basal NQO1 expression (NQ12) (FIG. 3b). Overexpression of NQO1 (EFNQ13) enhanced sensitivity to epirubicin while knockdown of NQO1 reduced cellular response, but only if p53 was functional (FIG. 3c,d). In contrast, after treatment with TNF, NQO1 levels determined the response regardless of p53 functionality in the U2OS-derived cell lines (FIG. 3e,f), resulting in enhanced response of NQO1-overexpressing cells and reduced response of NQO1-knockdown cells.

The differential roles of NQO1 and p53 were also observed in breast cancer cells MDA-MB157 (NQO1*1, PP) and MDA-MB231 (NQO1*2, SS), both lacking wild-type p53, which showed similar responses to epirubicin despite their different NQO1 genotypes (FIG. 3g). Reintroduction of NQO1 in MDA-MB231 had no effect on the response to epirubicin (FIG. 3h). In contrast, the NQO1-proficient MDA-MB157 cells responded better to TNF, consistent with the TNF-triggered pathway operating independently of p53 (FIGS. 3i and 3k).

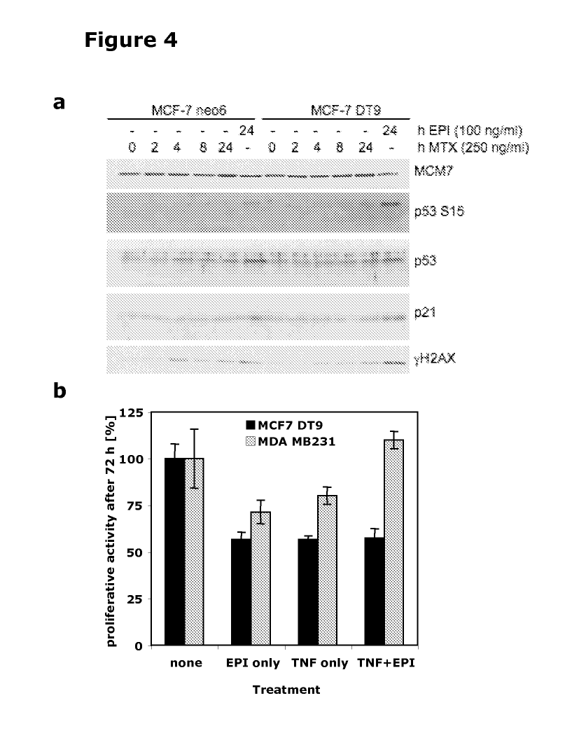

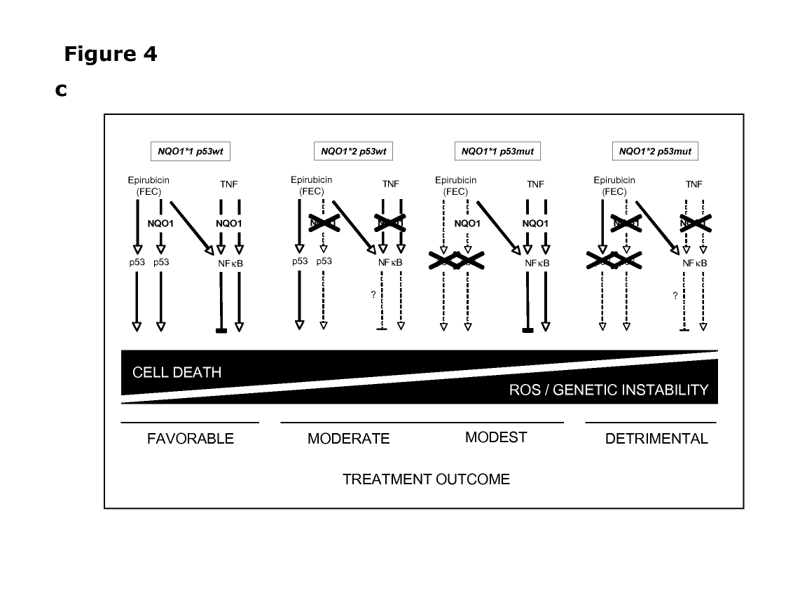

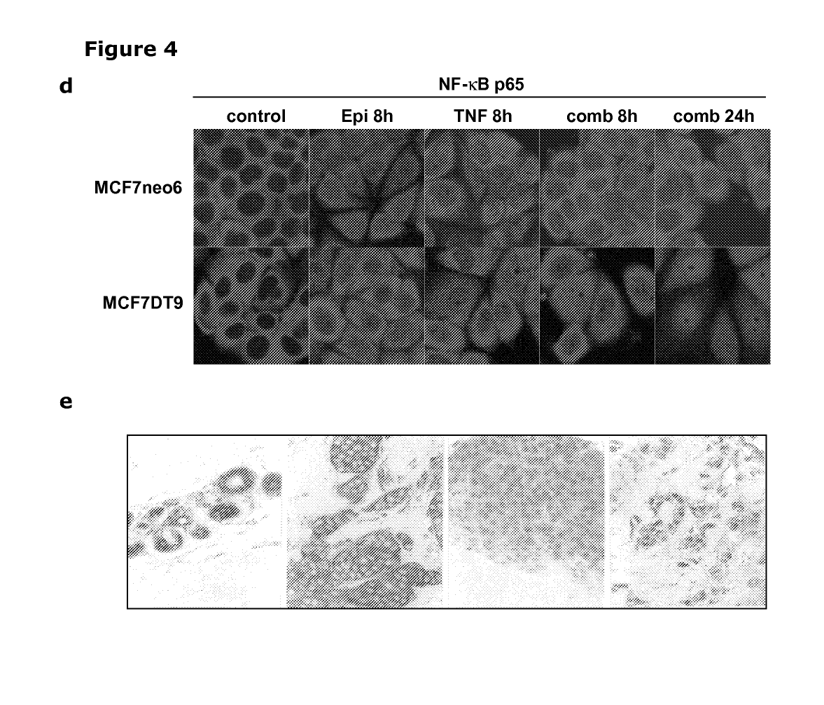

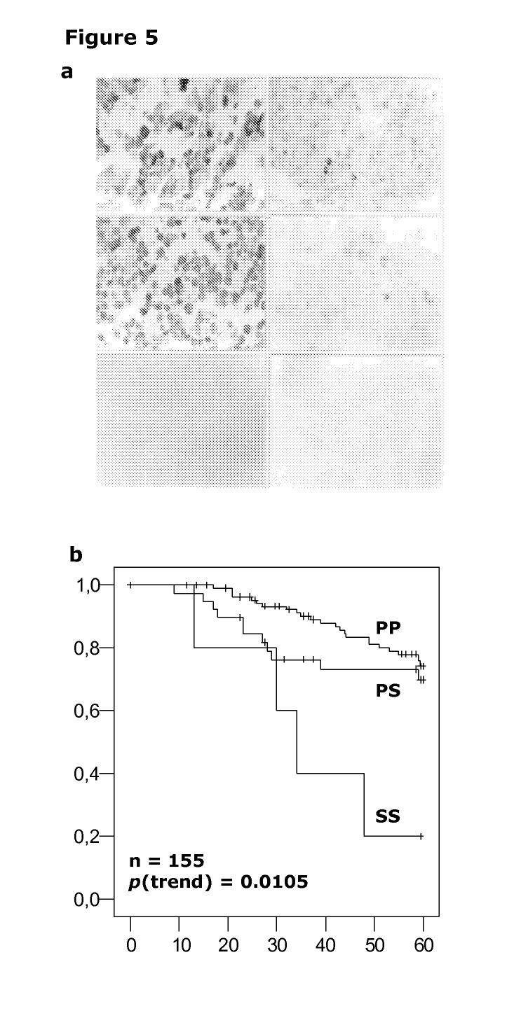

In order to investigate the effects of treatment with epirubicin, TNF and their combination on the NF-κB pathway we examined the cellular localization of the NF-κB subunit p65 in MCF7 cells (FIG. 4a). Nuclear translocation indicating activation of the NF-κB signaling pathway was detected after every treatment, however, the combined treatment had a prolonged activatory effect compared to the single treatment regimens.

Based on the suggested elevated serum levels of TNF in breast cancer patients (Perik et al. 2006; Berberoglu et al. 2004) it was studied in a subset of breast cancer patients (n=80) whether the NF-κB pathway is active using immunohistochemical staining of the tumors. Indeed, we detected nuclear localization of p65 (FIG. 4b) in about 25% of the investigated tumors even before adjuvant chemotherapy was initiated. In contrast, tissues from healthy controls showed exclusively cytosolic localization. These results indicate that some endogenous NF-κB-activating stimulus was present in a significant fraction of cases before therapy and render this additional pathway indeed clinically relevant.

### Example 8

### Combined Epirubicin/TNF Treatment does not Inhibit Proliferation of p53-Mutant, NQO1-Deficient Breast Cancer Cells

The differences in clinical outcome seen among the differentially treated patients with distinct NQO1 and p53 status led us to raise some testable predictions for responses in cultured cells. First, given the lack of association between NQO1 status and survival among methotrexate (CMF)-treated patients (Table 6), we hypothesized that unlike epirubicin, methotrexate may not activate the p53-p21 and/or TNF-NF-κB pathways. Consistent with this prediction, methotrexate is known to inhibit, rather than activate the cell death-inducing NF-κB mechanism (Majumdar et al., 2001), and our experiments with MCF7 cell lines showed an overall lower response of the p53/p21 pathway compared with epirubicin treatment, and no differences in cells with low versus high NQO1 expression (FIG. 4a).

Second, we argued that breast cancer cells with mutant p53 and the NQO1*2 (SS) genotype, closely mimicking the subset of patients with NQO1*2 (SS) genotype and p53-immunopositivity with the highest risk of death (Table 2), might be resistant even to a combined treatment with epirubicin and TNF. Indeed, whereas the p53-wildtype, NQO1-expressing MCF7 cells showed reduced proliferation in response to epirubicin alone, TNF alone, or a combined epirubicin/TNF treatment, proliferation of the p53-mutant, NQO1*2 MDA-MB231 cells was only modestly inhibited by either treatment alone. Most significantly, the concomitant treatment with epirubicin and TNF not only did not inhibit, but even slightly stimulated proliferation of these p53/NQO1 double-defective cells (FIG. 4b), thereby supporting the clinical data.

### Example 9

### NQO1*2 Homozygous Patients have Reduced Survival after Breast Cancer Metastasis

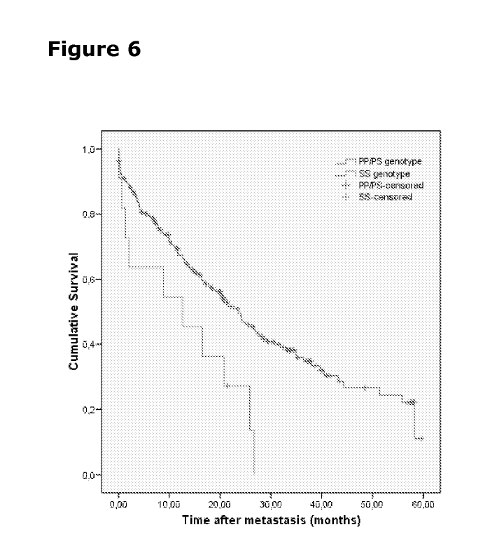

Anthracycline combination chemotherapies are the most effective and widely used regimens for the treatment of metastatic breast cancer (Fossati et al. 1998, A'Hern et al. 1993). If NQO1*2 confers cellular resistance to anthracyclines at a clinically significant level, one might expect to see a reduction in survival among NQO1*2 homozygous patients with metastatic breast cancer. Indeed, SS homozygous patients have a reduced rate of survival after diagnosis of metastasis, as indicated in the FIG. 6 by the Kaplan-Meier survival curve depicting the five-year survival of 227 patients after they have been diagnosed with metastatic breast cancer. This sample set includes all patients with metastatic breast cancer described in Example 1.

### Example 10

### The Use of the NQO1 Gene and its Gene Product

The present invention discloses for the first time the NQO1*2 genotype as a prognostic and predictive factor for cancer treatment, especially in breast cancer, using an in-depth statistical approach among incident cases. Its effect on breast cancer susceptibility, the clinical and histopathological characteristics of the tumors, as well as overall and metastasis-free survival of the subjects, using extensive, well characterized sample sets of sufficient size to provide adequate statistical power was analyzed. Furthermore, functional in vitro analyses were performed to validate and mechanistically support the genetic and clinicopathological findings.

An association between homozygous NQO1*2 and poor survival among 994 breast cancer patients, especially after anthracycline-based adjuvant chemotherapy with epirubicin (FEC) (5-year cumulative survival 0.17, 95% C.I. 0.00-0.47, p<0.0001) was shown. NQO1*2 homozygosity, combined with FEC treatment and p53 immunopositive tumors, was identified as an independent, highly significant predictor of poor outcome (RR of death 13.61, 95% CI-3.86-47.94, p<0.0001). Furthermore, response to epirubicin and TNF was impaired in NQO1*2 homozygous breast carcinoma cells and lymphoblasts derived from the patients. A model of defective apoptosis in homozygous NQO1*2 cells is proposed, characterized by impaired p53- and TNF/NF-κB mediated apoptosis and reduced epirubicin and TNF-induced cytotoxicity and NQO1 genotyping for subjects qualifying for anthracycline-based chemotherapy is recommended.

A highly significant association between NQO1*2 homozygosity and adverse breast cancer outcome as well as higher metastatic potential was detected. In particular, NQO1*2 predicts only 17% survival after anthracycline-based adjuvant chemotherapy with epirubicin (FEC), with even the most conservative estimates (upper 95% confidence interval) indicating only a 47% cumulative five-year survival for NQO1*2 homozygotes versus 67% (lower 95% confidence interval) among other genotypes in the FEC-treated group, indicating a dramatic difference. NQO1*2 is also associated with reduced survival among patients with p53-immunopositive tumors, with 20% cumulative 5-year survival.

Genetic and clinical observations are functionally validated and are mechanistically supported by in vitro studies of four complementary cell culture models where response to epirubicin, and TNF was analyzed in genetically modified cancer cells but also in non-malignant cell lines obtained from genotyped patients. Consistently, NQO1-deficient NQO1*2 cells (SS) were more resistant to epirubicin than the NQO1-proficient cells (NQO1*1), and enhanced levels of NQO1 rendered cells more sensitive to epirubicin and TNF treatment. Especially, NQO1 enhances TNF-mediated cell death in human breast cancer and sarcoma cell lines.

Based on the available literature and the present results, it could be proposed that NQO1 influences the outcome of epirubicin treatment probably through at least three mechanisms: the p53 tumor suppressor and TNF/NF-κB pathways and direct detoxification of reactive oxygen species (ROS) (FIG. 4c). Whereas the role of NQO1 in the TNF/NF-κB cascade remains to be understood mechanistically, the p53-related function likely reflects the NQO1-mediated protection of p53 from “degradation by default” via the 20S proteasome (Asher et al., 2001; 2002a; 200b). Contrary to the MDM2/ubiquitin-mediated degradation of p53 via the 26S proteasome, “degradation by default” does not require modification of p53 (Asher et al., 2005). This leads to lower-than-basal levels of p53 in cells lacking functional NQO1 (Asher et al., 2001), and explains the transient nature of the NQO1 effects on p53/p21 in our present experiments, later masked by the NQO1-independent predominant effects of the MDM2 pathway. Importantly, even the transient shortage of wild-type p53 observed in the epirubicin-treated, NQO1-deficient cells increases cancer cell survival in vitro, and this correlates with reduced survival of the patients after epirubicin-based chemotherapy.

In broader terms, the simplified functional model of the present invention suggests several scenarios that differentially affect responses to epirubicin in breast cancer cells (FIG. 4c). The cellular response to epirubicin is most favorable (causing maximum cancer cell death) when both p53 and NQO1 are normal. Less pronounced, yet still positive effects are seen with either NQO1 or p53 deficiency, consistent with partly linked and partly mutually independent roles of the two proteins in the parallel cell-death pathways (FIG. 4c). Importantly, the concomitant deficiency of both p53 and NQO1 appears to be detrimental for cellular responses to epirubicin treatment and survival of the breast cancer patients. This combination not only disables the two pro-apoptotic pathways, but it may even enhance cancer cell survival and/or promote progression of such therapy-resistant tumors (FIG. 1, see also Table 6). Such adverse effects may reflect enhanced genomic instability fueled by epirubicin-induced DNA damage in cells rendered highly tolerant of damaged DNA due to dysfunctional p53 and NQO1. Another mechanism that possibly contributes to enhanced cancer cell survival are the pro-survival (rather than pro-apoptotic) effects of the p53- and NQO1-independent branch of the NF-κB pathway that responds to the DNA damage-induced ATM kinase and NEMO, an upstream regulator of NF-κB (Kovalenko et al., 2006). Also, p53 transcriptionally represses those anti-apoptotic and proliferation-inducing capacities of NF-κB (Janssens et al., 2006). Last but not least, the anti-oxidant functions of both wild-type p53 (Sablina et al., 2005) and NQO1 (see Introduction) are no doubt important under the conditions of enhanced oxidative stress in cancer cells, and the combined lack of these detoxifying effects likely results in more pronounced ROS-induced DNA damage, enhanced genetic instability and further cancer progression (FIG. 4c). The fact that NQO1 is particularly required when p53 is aberrant is apparent also from the notion that patients with p53-immuno-positive tumors show reduced survival when they are NQO1 heterozygous (PS), compared with the NQO1 wild-type homozygotes (supplementary FIG. S1b). Although still inevitably simplified and partly speculative, this model (see FIG. 4c for details) is consistent with the clinical and experimental data.

Taken together, the clinical and functional findings suggested reduced epirubicin and TNF-induced cytotoxicity in NQO1*2 homozygous breast cancer, with a drastic reduction in survival among patients who have undergone treatment, preferably adjuvant—particularly epirubicin-based—chemotherapy. This can have an impact on a significant number of patients at the global population level, since some 4% of Caucasians and even up to 20% of Asian population are homozygous for NQO1*2 (Kelsey et al., 1997; Nioi et al., 2004) Annually, more than one million breast cancer cases are diagnosed worldwide (Parkin et al., 2005) and a significant proportion of these patients qualify for anthracycline-based treatment. Among such patients, NQO1 genotype provides a predictive factor for treatment. The NQO1 status may be used to provide predictive information also for other types of malignancies. The value of NQO1 as a candidate predictive factor in patients treated with other modalities that cause DNA breakage and/or trigger apoptotic response in a way analogous to epirubicin is studied.

In the present invention a NAD(P)H:Quinone oxidoreductase 1 (NQO1) gene, which carries a c.609C>T allele resulting a protein encoding P187S is used as the predictive marker. In a preferred embodiment of the present invention the method comprises the detection of the presence of a mutant or absence of normal or functional gene or gene product, including transcription or translation products. The invention is based on genotyping and phenotyping methods, applying techniques based on specific measurement of DNA, RNA or amino acid sequences or functionality. Examples of such sequence specific genotyping methods include but are not limited to a technique for single nucleotide polymorphism (SNP) detection and genotyping, such as restriction fragment length polymorphism PCR (RFLP-PCR), SSCP, allele specific hybridization, primer extension, allele specific oligonucleotide ligation or sequencing. The so called minisequencing method described in WO 91/13075 applies DNA polymerase for identifying SNPs may be used as well as methods applying reverse transcriptase for identifying SNPs.

A polymorphism in NQO1 is known to result in extremely limited amounts or a total lack of the enzyme and therefore the activity can be used to screen potential patients. It is known that homozygous carriers of the c.609C>T allele, often referred to as NQO1*2, have no measurable NQO1 activity, reflecting very low levels of the NQO1 P187S protein due to its rapid turnover via the ubiquitin proteasomal pathway (Siegel et al., 1999; 2001). Therefore, the genotype of a person may be determined indirectly through the determination of the phenotype by measuring the level of NQO1 activity. The NQO1 activity may be determined e.g. by using a substrate described in Beall et al., Cancer Res. 54:3196-3201 (1994) and Siegel et al., Mol. Pharmacol., 44:1128-1134 (1993), Siegel et al., Cancer Res., 50:7293-7300 (1990). In fact, AZQ failed to show any Beall et al., Mol. Pharmacol. 48:499-504 (1995), Ross et al., Cancer Metastasis Rev., 12:83-101 (1993).

The activity measurement thereby provides a useful method for measuring from a protein containing sample whether the subject would benefit from being excluded from a particular treatment or not. Reduced level or a total lack of the NQO1 enzyme in a sample can be determined also by methods, such as immunoblotting using a polyclonal or monoclonal antibody specific for NQO1 protein.

TABLE 1
Histopathological characterization of unselected
breast tumors according to NQO1 genotype.

| | | | | | | | | | |
|---|---|---|---|---|---|---|---|---|---|
| Category | Total | (%) | PP | (%) | PS | (%) | SS | (%) | p-value |

Tumor histology (n = 1,757)

| | | | | | | | | | |
|---|---|---|---|---|---|---|---|---|---|
| Ductal | 1,180 | (67.2%) | 796 | (68.1%) | 340 | (64.9%) | 44 | (68.8%) | ns |
| Lobular | 391 | (22.3%) | 253 | (21.6%) | 125 | (23.9%) | 13 | (20.3%) | |
| Other | 186 | (10.6%) | 120 | (10.3%) | 59 | (11.3%) | 7 | (10.9%) | |

Grade (n = 1,683)

| | | | | | | | | | |
|---|---|---|---|---|---|---|---|---|---|
| 1 | 479 | (28.5%) | 320 | (28.5%) | 142 | (28.5%) | 17 | (26.6%) | ns |
| 2 | 736 | (43.7%) | 486 | (43.4%) | 221 | (44.4%) | 29 | (45.3%) | |
| 3 | 468 | (27.8%) | 315 | (28.1%) | 135 | (27.1%) | 18 | (28.1%) | |

T (n = 1,744)

| | | | | | | | | | |
|---|---|---|---|---|---|---|---|---|---|
| 1 + 2 | 1,616 | (92.7%) | 1,068 | (92.1%) | 489 | (94.0%) | 59 | (92.2%) | ns |
| 3 + 4 | 128 | (7.3%) | 92 | (7.9%) | 31 | (6.0%) | 5 | (7.8%) | |

N (n = 1,734)

| | | | | | | | | | |
|---|---|---|---|---|---|---|---|---|---|
| negative | 943 | (54.4%) | 623 | (53.9%) | 285 | (55.3%) | 35 | (54.7%) | ns |
| positive | 791 | (45.6%) | 532 | (46.1%) | 230 | (44.7%) | 29 | (45.3%) | |

M (n = 1,667)

| | | | | | | | | | |
|---|---|---|---|---|---|---|---|---|---|
| negative | 1,601 | (96.0%) | 1,069 | (96.3%) | 477 | (96.0%) | 55 | (91.7%) | ns |
| positive | 66 | (4.0%) | 41 | (3.7%) | 20 | (4.0%) | 5 | (8.3%) | |

ER (n = 1,723)

| | | | | | | | | | |
|---|---|---|---|---|---|---|---|---|---|
| negative | 314 | (18.2%) | 204 | (17.8%) | 95 | (18.6%) | 15 | (23.8%) | ns |
| positive | 1,409 | (81.8%) | 944 | (82.2%) | 417 | (81.4%) | 48 | (76.2%) | |

PgR (n = 1,723)

| | | | | | | | | | |
|---|---|---|---|---|---|---|---|---|---|
| negative | 599 | (34.8%) | 385 | (33.5%) | 188 | (36.7%) | 26 | (41.3%) | ns |
| positive | 1,124 | (65.2%) | 763 | (66.5%) | 324 | (63.3%) | 37 | (58.7%) | |

p53 IHC (n = 1,350)

| | | | | | | | | | |
|---|---|---|---|---|---|---|---|---|---|
| negative | 1,026 | (76.0%) | 690 | (76.2%) | 306 | (77.3%) | 30 | (62.5%) | 0.026 |
| positive | 324 | (24.0%) | 216 | (23.8%) | 90 | (22.7%) | 18 | (37.5%) | |

P-values have been calculated for SS (NQO1*2) homozygotes versus other genotypes; ns indicates a statistically non-significant p-value. Whenever a cell value was 5 or less, Fisher's exact test was used instead of the Chi-square test. Cases of carcinoma in situ were excluded from the analysis. Abbreviations: T, tumor diameter; N, nodal status; M, distant metastases; ER, estrogen receptor; PgR, progesterone receptor; P53 ICH, p53 immunohistochemistry

TABLE 2
Interactions between NQO1 genotype, p53 immunohistochemistry
(IHC) and FEC treatment status emerge as independent prognostic
factors in multivariate survival analysis.

| | | |
|---|---|---|
| Covariate | p-value | R.R. (95% CI) |

A. Treatment not included

| | | | |
|---|---|---|---|
| T | 0.001 | 3.07 | (1.59-5.92) |
| N | <0.001 | 4.69 | (2.32-9.49) |
| M | <0.001 | 5.11 | (2.45-10.66) |
| PgR | <0.001 | 0.31 | (0.17-0.54) |
| P53 IHC | <0.001 | 3.34 | (1.91-5.81) |
| [NQO1*2 & p53 IHC] | 0.018 | 3.65 | (1.25-10.67) |

B. Treatment included

| | | | |
|---|---|---|---|
| T | <0.001 | 3.47 | (1.80-6.71) |
| N | <0.001 | 4.54 | (2.24-9.21) |
| M | <0.001 | 5.15 | (2.48-10.69) |
| PgR | <0.001 | 0.28 | (0.16-0.49) |
| p53 IHC | <0.001 | 3.36 | (1.95-5.79) |
| [NQO1*2 & FEC] | 0.001 | 12.69 | (3.68-43.78) |

Optimized Cox's proportional hazards model of predictive factors in breast cancer, independently of adjuvant chemotherapy (a) and with the type of adjuvant chemotherapy factored in (b), including interactions between two variables. All variables in the output are binary and categorical (see Table 4); RR represents the average risk ratio of death at any given point during the follow-up time among patients positive for the characteristic, within the context of this model. To qualify as positive for the interaction terms, a patient must be positive for all of its constituents; patients with missing data have been excluded from the analysis. n of valid cases = 685. Abbreviations: FEC, 5-fluorouracil (5-FU) + epirubicin + cyclophosphamide; T, tumor diameter; N, nodal status; M, distant metastases; ER, estrogen receptor; PgR, progesterone receptor; P53 ICH, p53 immunohistochemistry

TABLE 3
Descriptive statistics of the sample
set used in the survival analyses.

| Category | Definition | Value (Freq.) |
|---|---|---|
| Age at Diagnosis (years) | Minimum | 22.3 |
| | Maximum | 95.6 |
| | Mean | 56.7 |
| | Standard Deviation | 12.4 |
| Followup time (months) | Minimum | 6.0 |
| | Maximum | 123.5 |
| | Mean | 64.7 |
| | Standard Deviation | 25.2 |
| p53 immunohistochemistry | negative | 607 (61.0%) |
| | positive | 155 (15.6%) |
| | data unavailable | 232 (23.3%) |
| Vital status | alive | 835 (84.0%) |
| | dead | 159 (16.0%) |
| Treatment for primary | Radiation therapy | 862 (86.7%) |
| breast cancer* | Endocrine therapy | 457 (49.9%) |
| | Adjuvant chemotherapy | 380 (38.2%) |
| | None/Surgical only | 42 (4.2%) |
| Type of adjuvant | FEC | 164 (16.5%) |
| chemotherapy | CMF | 193 (19.4%) |
| | Other | 23 (2.3%) |
| | None | 614 (61.8%) |

The total number of the sample set (incident cases with NQO1 P187S genotype and sufficient followup data available) is 994. Abbreviations: FEC, 5-fluorouracil (5-FU) + epirubicin + cyclophosphamide; CMF, cyclophosphamide + methotrexate + fluorouracil 5-FU
*The treatment types are not mutually exclusive, hence the percentages do not add up to 100%.

TABLE 4
Variables included in the multivariate
Cox's proportional hazards analyses.

| | |
|---|---|
| Variable | Coding |

a. Treatment not included

| | |
|---|---|
| T | T1 vs T2-4 |
| M | positive vs negative |
| N | positive vs negative |
| ER | positive vs negative |
| PgR | positive vs negative |
| p53 IHC | positive vs negative |
| Grade | 1, 2, 3 (categorical) |
| NQO1*2 | PP/PS vs SS |
| [NQO1*2 & p53 IHC] | (interaction) |

b. Treatment included

| | |
|---|---|
| T | T1 vs T2-4 |
| M | positive vs negative |
| N | positive vs negative |
| ER | positive vs negative |
| PgR | positive vs negative |
| p53 IHC | positive vs negative |
| Grade | 1, 2, 3 (categorical) |
| NQO1*2 | PP/PS vs SS |
| FEC | treated vs non-treated |
| [NQO1*2 & FEC] | (interaction) |
| [FEC & p53 IHC] | (interaction) |
| [NQO1*2 & p53 IHC] | (interaction) |

All of these variables were available for the Cox's regression optimization algorithm; in the final models, as displayed in Table 2, only the variables that remain in the best fit model after the optimization process are displayed. Abbreviations: FEC, 5-fluorouracil (5-FU) + epirubicin + cyclophosphamide; T, tumor diameter; N, nodal status; M, distant metastases; ER, estrogen receptor; PgR, progesterone receptor; P53 ICH, p53 immunohistochemistry.

TABLE 5
NQO1 P187S genotype frequencies by sample set and age group.

| Sample set | | Total | (%) | PP | (%) | PS | (%) | SS | (%) | Sig. |
|---|---|---|---|---|---|---|---|---|---|---|
| Unselected | Age <50 | 476 | (100.0%) | 328 | (68.9%) | 134 | (28.2%) | 14 | (2.9%) | ns |
| | Age ≧50 | 1,218 | (100.0%) | 796 | (65.4%) | 374 | (30.7%) | 48 | (3.9%) | ns |
| | All | 1,694 | (100.0%) | 1,124 | (66.4%) | 508 | (30.0%) | 62 | (3.7%) | ns |
| Familial | Age <50 | 278 | (100.0%) | 187 | (67.3%) | 84 | (30.2%) | 7 | (2.5%) | ns |
| | Age ≧50 | 527 | (100.0%) | 347 | (65.8%) | 169 | (32.1%) | 11 | (2.1%) | ns |
| | All | 805 | (100.0%) | 534 | (66.3%) | 253 | (31.4%) | 18 | (2.2%) | ns |
| Controls | Age <50 | 457 | (100.0%) | 310 | (67.8%) | 133 | (29.1%) | 14 | (3.1%) | — |
| | Age ≧50 | 241 | (100.0%) | 159 | (66.0%) | 72 | (29.9%) | 10 | (4.1%) | — |
| | All | 698 | (100.0%) | 469 | (67.2%) | 205 | (29.4%) | 24 | (3.4%) | — |

Genotype frequencies were compared between the population controls and subgroups of cases using a Chi-square test of independence; ns denotes a non-significant p-value (no association).

TABLE 6
Overall and metastasis-free survival statistics among subgroups stratified by treatment and p53 immunohistochemistry.

| | | Cumulative 5-year overall survival | | | | | Cumulative 5-year metastasis-free survival | | | | |
|---|---|---|---|---|---|---|---|---|---|---|---|
| Subgroup | n | PP/PS | (95% C.I.) | SS | (95% C.I.) | p-value | PP/PS | (95% C.I.) | SS | (95% C.I.) | p-value |
| All | 994 | 0.86 | (0.84-0.88) | 0.65 | (0.47-0.82) | 0.0006 | 0.81 | (0.78-0.84) | 0.73 | (0.57-0.89) | 0.1562 |
| Endocrine treatment given | 352 | 0.84 | (0.80-0.88) | 0.74 | (0.49-0.99) | 0.2835 | 0.78 | (0.73-0.83) | 0.74 | (0.49-0.99) | 0.5882 |
| No endocrine treatment | 479 | 0.90 | (0.87-0.93) | 0.82 | (0.59-1.00) | 0.3896 | 0.86 | (0.83-0.89) | 1.00 | (n/a) | 0.2069 |
| No chemotherapy | 615 | 0.89 | (0.86-0.92) | 0.79 | (0.60-0.97) | 0.1375 | 0.85 | (0.82-0.88) | 0.90 | (0.77-1.00) | 0.6821 |
| Chemotherapy given | 379 | 0.81 | (0.77-0.85) | 0.40 | (0.10-0.70) | 0.0002 | 0.74 | (0.69-0.79) | 0.40 | (0.10-0.70) | 0.0028 |
| FEC treatment | 163 | 0.75 | (0.67-0.83) | 0.17 | (0.00-0.47) | <0.0001 | 0.69 | (0.61-0.77) | 0.17 | (0.00-0.47) | 0.0007 |
| CMF treatment | 193 | 0.86 | (0.81-0.91) | 0.75 | (0.33-1.00) | 0.5691 | 0.78 | (0.72-0.84) | 0.75 | (0.33-1.00) | 0.8114 |
| p53 IHC positive | 154 | 0.73 | (0.65-0.81) | 0.20 | (0.00-0.55) | 0.0007 | 0.67 | (0.59-0.75) | 0.20 | (0.00-0.55) | 0.0030 |
| p53 IHC negative | 607 | 0.90 | (0.87-0.93) | 1.00 | (n/a) | 0.2386 | 0.84 | (0.81-0.87) | 0.94 | (0.82-1.00) | 0.3835 |

Patients who received FEC treatment have been excluded from the endocrine treatment based subgroups. Confidence intervals for cumulative survival after five years of follow-up are provided, along with p-values from the log-rank test between SS (NQO1*2) homozygotes vs. other (PP/PS) genotypes.

### REFERENCES

WO 2005/119260
WO 2005/024067
WO 2005/098037
WO 02052044
WO 2004058153
WO 2006035273
US 2003158251
US 20010034023
Ahn, K. S., Sethi, G., Jain, A. K., Jaiswal, A. K. & Aggarwal, B. B. Genetic deletion of NAD(P)H:quinone oxidoreductase 1 abrogates activation of nuclear factor-kappaB, IkappaBalpha kinase, c-Jun N-terminal kinase, Akt, p38, and p44/42 mitogen-activated protein kinases and potentiates apoptosis. J. Biol. Chem. 281, 19798-19808 (2006).
Anwar, A., et al. Interaction of human NAD(P)H:quinone oxidoreductase 1 (NQO1) with the tumor suppressor protein p53 in cells and cell-free systems. J. Biol. Chem. 278, 10368-10373 (2003).
Asher, G., Lotem, J., Cohen, B., Sachs, L. & Shaul, Y. Regulation of p53 stability and p53-dependent apoptosis by NADH quinone oxidoreductase 1. Proc. Nat. Acad. Sci. USA 98, 1188-1193 (2001).
Asher, G., Lotem, J., Kama, R., Sachs, L. & Shaul, Y. NQO1 stabilizes p53 through a distinct pathway. Proc. Nat. Acad. Sci. USA 99, 3099-3104 (2002a).
Asher, G., Lotem, J., Sachs, L., Kahana, C. & Shaul, Y. Mdm-2 and ubiquitin-independent p53 proteasomal degradation regulated by NQO1. Proc. Nat. Acad. Sci. USA 99, 13125-13130 (2002b).
Asher, G., Bercovich, Z., Tsvetkov, P., Shaul, Y. & Kahana, C. 20S proteasomal degradation of ornithine decarboxylase is regulated by NQO1. Mol. Cell. 17, 645-655 (2005).
Asher, G. & Shaul, Y. p53 proteasomal degradation: poly-ubiquitination is not the whole story. Cell cycle 4, 1015-1018 (2005).
Barragan et al. The GST deletions and NQO1*2 polymorphism confers interindividual variability of response to treatment in patients with acute myeloid leukemia. Leuk. Res. 31: 947-953 (2007).
Berberoglu U, Yildirim E, Celen O, Serum levels of tumor necrosis factor alpha correlate with response to neoadjuvant chemotherapy in locally advanced breast cancer. Int J Biol Markers 2004; 19(2):130-4.
Beyer, R. E., et al. The role of DT-diaphorase in the maintenance of the reduced antioxidant form of coenzyme Q in membrane systems. Proc. Nat. Acad. Sci. USA 93, 2528-2532 (1996).
Chao, C., Zhang, Z. F., Berthiller, J., Boffetta, P. & Hashibe, M. NAD(P)H:quinone oxidoreductase 1 (NQO1) Pro187Ser polymorphism and the risk of lung, bladder, and colorectal cancers: a meta-analysis. Cancer Epidemiol. Biomarkers Prev. 15, 979-987 (2006).
Codony-Servat J, Tapia M A, Bosch M, Oliva C, Domingo-Domenech J, Mellado B, Rolfe M, Ross J S, Gascon P, Rovira A, Albanell J. Differential cellular and molecular effects of bortezomib, a proteasome inhibitor, in human breast cancer cells. Mol Cancer Ther. 5, 665-75 (2006).
Eerola, H., Blomqvist, C., Pukkala, E., Pyrhonen, S. & Nevanlinna, H. Familial breast cancer in southern Finland: how prevalent are breast cancer families and can we trust the family history reported by patients? Eur. J. Cancer 36, 1143-1148 (2000).
Estevam, F. R., Augusto, S. F., Rodrigues, S. A., Pinheiro, M. R. & Monteiro, A. F. Apoptosis and production of TNF-alpha by tumor-associated inflammatory cells in histological grade III breast cancer. Cancer Immunol. Immunother. 54, 671-676 (2005).
Fleming et al. Clinical significance of a NAD(P)H: quinine oxidoreductase 1 polymorphism in patients with disseminated peritoneal cancer receiving intraperitoneal hyperthermic chemotherapy with mitomycin C. Pharmacogenetics 12: 31-37 (2002).
Fowke, J. H., et al. Oral contraceptive use and breast cancer risk: modification by NAD(P)H:quinone oxoreductase (NQO1) genetic polymorphisms. Cancer Epidemiol. Biomarkers Prev. 13, 1308-1315 (2004).
Garte, S., et al. Biomarkers of exposure and effect in Bulgarian petrochemical workers exposed to benzene. Chem. Biol. Interact. 153-154, 247-251 (2005).
Goldberg et al. Role of a DT-diaphorase mutation in the response of anal canal carcinoma to radiation, 5-fluorouracil, and mitomycin C. Int. J. Radiat. Oncol. Biol. Phys. 42: 331-334 (1998).
Goldhirsch, A., Glick, J. H., Gelber, R. D., Coates, A. S. & Senn, H. J. Meeting highlights: International Consensus Panel on the Treatment of Primary Breast Cancer. Seventh International Conference on Adjuvant Therapy of Primary Breast Cancer. J. Clin. Oncol. 19, 3817-3827 (2001).
Goode, E. L., et al. Effect of germ-line genetic variation on breast cancer survival in a population-based study. Cancer res. 62, 3052-7 (2002).
Iskander, K., et al. Lower induction of p53 and decreased apoptosis in NQO1-null mice lead to increased sensitivity to chemical-induced skin carcinogenesis. Cancer Res. 65, 2054-2058 (2005).
Iskander, K., Li, J., Han, S., Zheng, B. & Jaiswal, A. K. NQO1 and NQO2 regulation of humoral immunity and autoimmunity. J. Biol. Chem. 281, 30917-30924 (2006).
Janssens, S. & Tschopp, J. Signals from within: the DNA-damage-induced NF-kappaB response. Cell Death Differ. 13, 773-84 (2006).
Jenkins G J, Mikhail J, Alhamdani A, Brown T, Caplin S, Manson J, Bowden R, Toffazal N, Griffiths P, Parry J, Baxter J. Immunohistochemical study of NF-kB activity and IL-8 abundance in oesophageal adenocarcinoma; a useful strategy for monitoring these biomarkers. J Clin Pathol. 2007
Kastan, M B. Wild-Type p53: Tumors Can't Stand It. Cell; 128:837-40 (2007).
Kelsey, K. T., et al. Ethnic variation in the prevalence of a common NAD(P)H quinone oxidoreductase polymorphism and its implications for anti-cancer chemotherapy. Br. J. Cancer 76, 852-854 (1997).
Kilpivaara, O., et al. Correlation of CHEK2 protein expression and c.1100delC mutation status with tumor characteristics among unselected breast cancer patients. Int. J. Cancer 113, 575-580 (2005).
Kovalenko, A. & Wallach, D. If the prophet does not come to the mountain: dynamics of signaling complexes in NF-kappaB activation. Mol. Cell. 22, 433-436 (2006).
Krajinovic, M., Sinnett, H., Richer, C., Labuda, D. & Sinnett, D. Role of NQO1, MPO and CYP2E1 genetic polymorphisms in the susceptibility to childhood acute lymphoblastic leukemia. Int. J. Cancer 97, 230-236 (2002a).
Krajinovic, M., et al. Polymorphisms in genes encoding drugs and xenobiotic metabolizing enzymes, DNA repair enzymes, and response to treatment of childhood acute lymphoblastic leukemia. Clin. Cancer Res. 8, 802-810 (2002).
Larson, R. A., et al. Prevalence of the inactivating 609C-->T polymorphism in the NAD(P)H:quinone oxidoreductase (NQO1) gene in patients with primary and therapy-related myeloid leukemia. Blood 94, 803-807 (1999).
Lavin, M. F. & Gueven, N. The complexity of p53 stabilization and activation. Cell death and differentiation 13, 941-950 (2006).
Long, D. J., 2nd, et al. NAD(P)H:quinone oxidoreductase 1 deficiency increases susceptibility to benzo(a)pyrene-induced mouse skin carcinogenesis. Cancer Res. 60, 5913-5915 (2000).
Lukas, J. & Bartek, J. Immunoprecipitation of proteins under nondenaturing conditions. Cell biology: a laboratory handbook, 2nd edition 4, 489-494. (Celis, J. E. (ed), Academic Press, London, United Kingdom, 1998).
Mailand, N., et al. Rapid destruction of human Cdc25A in response to DNA damage. Science 288, 1425-1429 (2000).
Majumdar, S & Aggarwal, B. B. Methotrexate suppresses NF-kappaB activation through inhibition of IkappaBalpha phosphorylation and degradation. J. Immunol. 167, 2911-20 (2001).
Martin A M, Weber B L. Genetic and hormonal risk factors in breast cancer. J Natl Cancer Inst 2000; 92(14):1126-35.
Menzel, H. J., et al. Association of NQO1 polymorphism with spontaneous breast cancer in two independent populations. Br. J. Cancer 90, 1989-1994 (2004).
Miki, Y., et al. A strong candidate for the breast and ovarian cancer susceptibility gene BRCA1. Science 266, 66-71 (1994).
Naoe, T., et al. Analysis of genetic polymorphism in NQO1, GST-M1, GST-T1, and CYP3A4 in 469 Japanese patients with therapy-related leukemia/myelodysplastic syndrome and de novo acute myeloid leukemia. Clin. Cancer Res. 6, 4091-4095 (2000).
Nioi, P. & Hayes, J. D. Contribution of NAD(P)H:quinone oxidoreductase 1 to protection against carcinogenesis, and regulation of its gene by the Nrf2 basic-region leucine zipper and the arylhydrocarbon receptor basic helix-loop-helix transcription factors. Mutat. Res. 555, 149-171 (2004).
Parkin, D. M., Bray, F., Ferlay, J. & Pisani, P. Global cancer statistics, 2002. CA Cancer J. Clin. 55, 74-108 (2005).
Perik, P. J., et al. Circulating apoptotic proteins are increased in long-term disease-free breast cancer survivors. Acta Oncol. 45, 175-183 (2006).
Pritchard, K. I., et al. HER2 and responsiveness of breast cancer to adjuvant chemotherapy. N. Engl. J. Med. 354, 2103-11 (2006).
Ross et al. NAD(P)H:quinone oxidoreductase 1 (NQO1): chemoprotection, bioactivation, gene regulation and genetic polymorphisms. Chem. Biol. Interact. 129: 77-97 (2000).
Rothman, N., et al. Benzene poisoning, a risk factor for hematological malignancy, is associated with the NQO1 609C-->T mutation and rapid fractional excretion of chlorzoxazone. Cancer Res. 57, 2839-2842 (1997).
Sablina, A. A., et al. The antioxidant function of the p53 tumor suppressor. Nat. Med. 11, 1306-13 (2005).
Sarmanova, J., et al. Breast cancer: role of polymorphisms in biotransformation enzymes. Eur. J. Hum. Genet. 12, 848-854 (2004).
Siegel, D., Bolton, E. M., Burr, J. A., Liebler, D. C. & Ross, D. The reduction of alpha-tocopherolquinone by human NAD(P)H: quinone oxidoreductase: the role of alpha-tocopherolhydroquinone as a cellular antioxidant. Mol. Pharmacol. 52, 300-305 (1997).
Siegel, D., McGuinness, S. M., Winski, S. L. & Ross, D. Genotype-phenotype relationships in studies of a polymorphism in NAD(P)H:quinone oxidoreductase 1. Pharmacogenetics 9, 113-121 (1999).
Siegel, D., et al. Rapid polyubiquitination and proteasomal degradation of a mutant form of NAD(P)H:quinone oxidoreductase 1. Mol. Pharmacol. 59, 263-268 (2001).
Siegel, D., et al. NAD(P)H:quinone oxidoreductase 1: role as a superoxide scavenger. Mol. Pharmacol. 65, 1238-1247 (2004).
Shi et al. High throughput genotyping for the detection of a single nucleotide polymorphism in NAD(P)H quinine oxidoreductase (DT diaphorase) using TaqMan probes. Mol. Pathol. 52: 295-299 (1999).
Siemankowski, L. M., Morreale, J., Butts, B. D. & Briehl, M. M. Increased tumor necrosis factor-alpha sensitivity of MCF-7 cells transfected with NAD(P)H:quinone reductase. Cancer Res. 60, 3638-3644 (2000).
Smith, M. T., et al. Low NAD(P)H:quinone oxidoreductase 1 activity is associated with increased risk of acute leukemia in adults. Blood 97, 1422-1426 (2001).
Smith M T, Wang Y, Skibola C F, et al. Low NAD(P)H:quinone oxidoreductase activity is associated with increased risk of leukemia with MLL translocations in infants and children. Blood 2002; 100(13):4590-3.
Syrjakoski, K., et al. Population-based study of BRCA1 and BRCA2 mutations in 1035 unselected Finnish breast cancer patients. J. Natl. Cancer Inst. 92, 1529-1531 (2000).
Tommiska, J., et al. Breast cancer patients with p53 Pro72 homozygous genotype have a poorer survival. Clin. Cancer Res. 11, 5098-5103 (2005).
Vahteristo, P., Eerola, H., Tamminen, A., Blomqvist, C. & Nevanlinna, H. A probability model for predicting BRCA1 and BRCA2 mutations in breast and breast-ovarian cancer families. Br. J. Cancer 84, 704-708 (2001).
Wiemels, J. L., et al. A lack of a functional NAD(P)H:quinone oxidoreductase allele is selectively associated with pediatric leukemias that have MLL fusions. United Kingdom Childhood Cancer Study Investigators. Cancer Res. 59, 4095-4099 (1999).
Winski, S. L., et al. Relationship between NAD(P)H:quinone oxidoreductase 1 (NQO1) levels in a series of stably transfected cell lines and susceptibility to antitumor quinones. Biochem. Pharmacol. 61, 1509-1516 (2001).
Wooster, R., et al. Identification of the breast cancer susceptibility gene BRCA2. Nature 378, 789-792 (1995).
Wu, Z. H., Shi, Y., Tibbetts, R. S. & Miyamoto, S. Molecular linkage between the kinase ATM and NF-kappaB signaling in response to genotoxic stimuli. Science 311, 1141-6 (2006).
Xu, Y., et al. p53 Codon 72 polymorphism predicts the pathologic response to neoadjuvant chemotherapy in patients with breast cancer. Clin. Cancer Res. 11, 7328-7333 (2005).

1. A method for selecting a cancer therapy based on subject's genetic background, wherein the method comprises the steps of determining the presence of a mutant or non-functional NAD(P)H:Quinone oxidoreductase 1, NQO1, gene or gene product, or absence of a normal or functional NQO1 gene or gene product from a sample of the subject comprising healthy or tumor cells before the onset of a chemotherapy, wherein said NQO1 gene carries a change in a nucleotide sequence; and classifying subjects in at least two subsets wherein one subset having a normal or functional NQO1 gene may be treated with cancer therapy and another subset having a mutant or non-functional NQO1 gene would benefit from being excluded from said cancer therapy.

2. The method according to claim 1, wherein the absence of a normal or functional NQO1 gene or gene product from the sample of the subject due to homozygous, hemizygous or other genetic or genomic alterations indicates that the subject would benefit from being excluded from said cancer therapy.

3. The method according to claim 1 wherein the NQO1 gene carries a change of one or more nucleotides resulting in a non-functional NQO1 gene.

4. The method according to claim 1, wherein the NQO1 gene carries a change in the nucleotide sequence corresponding to the cytosine to thymine substitution at position 609 of the polynucleotide sequence in NCBI sequence ID:J03934.1 or refSNP ID:rs1800566 set forth in SEQ ID NO:4 comprising a c.609C>T allele or NQO1*2 polymorphism, thereby resulting in the amino acid change of proline to serine at position 187, P187S, of the encoded gene product.

5. The method according to claim 1, wherein the NQO1 gene in the tumor cells is non-functional or the normal gene or gene product is absent due to homozygous, hemizygous or other genetic or genomic alterations.

6. The method according to claim 3, wherein a change in a nucleotide sequence is in linkage disequilibrium to position 609 of the polynucleotide sequence in NCBI sequence ID:J03934.1 or refSNP ID:rs1800566 set forth in SEQ ID NO:4 or to any other change of one or more nucleotides in said polynucleotide sequence resulting in a similar functional effect.

7. The method according to claim 4, wherein two copies of the c.609C>T allele are present in the subject indicating that the subject is a homozygous carrier of the c.609C>T allele and benefits from being excluded from the cancer therapy.

8. The method according to claim 4, wherein one copy of the c.609C>T allele is present in the tumor with loss or inactivation of the other allele indicating that the tumor cells are hemizygous for the c.609C>T allele and the subject benefits from being excluded from the cancer therapy.

9. The method according to claim 4, wherein the method comprises determining the identity of nucleotides in the nucleotide position c.609; and classifying the subject to a subset having a mutant or non-functional NQO1 gene if the T allele is present in both copies in the c.609 position, and to a subset having a normal or functional NQO1 gene if one of the alleles present in the c.609 position is C.

10. The method according to claim 1, wherein the cancer therapy comprises chemotherapy.

11-14. (canceled)

15. The method according to claim 1, wherein the cancer therapy comprises early curative therapy.

16. The method according to claim 1, wherein the cancer therapy comprises treatment of metastatic cancer.

17. The method according to claim 1, wherein the subject suffers from a cancer or a malignancy.

18. The method according to claim 17, wherein said cancer or malignancy comprises breast cancer, lung, bladder, prostatic, ovarian, pancreatic, gastric or colorectal cancer, cancer of the large intestine, non-Hodgkin's lymphoma, head neck cancer, large cell lung carcinoma, small cell lung carcinoma or soft tissue sarcoma or children's tumor.

19-20. (canceled)

21. The method according to claim 1, wherein the subject belonging to a subset of subjects that would benefit from being excluded from said cancer therapy is a breast cancer patient homozygous for the c.609C>T allele or NQO1*2 polymorphism of NQO1 gene, or any other change of one or more nucleotides in said polynucleotide sequence resulting in a similar functional effect, or a patient having tumor cells hemizygous for the c.609C>T allele or NQO1*2 polymorphism, or any other change of one or more nucleotides in said polynucleotide sequence resulting in a similar functional effect, and said cancer therapy is an anthracyclin-based adjuvant chemotherapy.

22. The method according to claim 1, wherein the subject belonging to a subset of subjects that would benefit from being excluded from said cancer therapy is a breast cancer patient heterozygous for the c.609C>T allele or NQO12 polymorphism or any other change of one or more nucleotides resulting in a similar functional effect of NQO1 gene and wherein the cancer comprises a p53 immunopositive tumor and said cancer therapy is an anthracyclin-based adjuvant chemotherapy.

23. (canceled)

24. The method according to claim 1, wherein the presence of a mutant or non functional or absence of a normal or functional NQO1 gene or gene product is determined from a sample comprising a DNA, or RNA, or protein or a fragment thereof, originating from the subject and representing an inherited genotype of the subject, or a genotype of a tumor.

25.-29. (canceled)

30. A method for treating a subject suffering from cancer or malignancy, comprising determining the presence of a mutant or non-functional NQO1 gene or gene product, or absence of a normal or functional NQO1 gene or gene product from a sample of the subject; and determining the proper therapy for said subject based on results of the genotype determination, wherein in the absence of a normal or functional NQO1 gene the subject is excluded from a cancer therapy.

31. A method for optimizing clinical trial design for selecting a cancer therapy based on subject's genetic background, wherein the method comprises determining the presence of a mutant or non-functional NQO1 gene or gene product, or absence of a normal or functional NQO1 gene or gene product from a sample of the subject; and allowing classification of the subjects in at least two subsets, wherein one subset having a normal or functional NQO1 gene may be treated with cancer therapy and another subset having a mutant or non-functional NQO1 gene would benefit from being excluded from said cancer therapy.

32. A method for selecting a cancer therapy for treatment of metastatic cancer based on subject's genetic background, wherein the method comprises the steps of determining the presence of a mutant or non-functional NQO1 gene or gene product or absence of a normal or functional NQO1 gene or gene product from a sample of the subject comprising healthy or tumor cells wherein said NQO1 gene carries a change in a nucleotide sequence; and classifying subjects in at least two subsets wherein one subset having a normal or functional NQO1 gene may be treated with cancer therapy and another subset having a mutant or non-functional NQO1 gene would benefit from being excluded from said cancer therapy.
